(19) United States
(12) Patent Application Publication
(10) Pub. No.: US 2023/0218652 A1
(43) Pub. Date: Jul. 13, 2023

(54) ANTIFUNGAL PRODRUGS

(71) Applicants: CENTRE NATIONAL DE LA RECHERCHE SCIENTIFIQUE, PARIS (FR); NANTES UNIVERSITÉ, NANTES (FR)

(72) Inventors: ARNAUD TESSIER, ORVAULT (FR); PATRICE LE PAPE, VERTOU (FR); JACQUES LEBRETON, NANTES (FR); FABRICE PAGNIEZ, LA CHAPELLE HEULIN (FR); GILLES-OLIVIER GRATIEN, NANTES (FR)

(21) Appl. No.: 17/927,941

(22) PCT Filed: May 28, 2021
(86) PCT No.: PCT/EP2021/064441

§ 371 Date: Nov. 28, 2022

(30) Foreign Application Priority Data
May 29, 2020 (EP) 20305562.9

(51) Int. Cl. A61K 31/7048; A61P 31/10

(52) U.S. Cl. CPC A61K 31/7048; A61P 31/10

(57) ABSTRACT

The invention relates to an antifungal prodrug which comprises an antifungal moiety which is linked to a trigger moiety by means of a self-immolative spacer. The trigger moiety is selected from glycosyl residues and oligosaccharides, stabilizes the self-immolative spacer and is cleavable by a pathogen hydrolytic enzyme which is preferably an extracellular glycosidase (EC 3.2.1). When the trigger moiety is cleaved by the pathogen hydrolytic enzyme, the self-immolative spacer undergoes a spontaneous degradation so as to release the antifungal moiety. The invention also relates to pharmaceutical compositions comprising said prodrug and to its use in the treatment of infectious diseases.

### FIELD OF THE INVENTION

The invention relates to the treatment of infectious diseases, in particular fungal and parasitic diseases.

### BACKGROUND OF THE INVENTION

As of today, more than 300 species of opportunistic fungi capable of infecting humans and animals have been identified. Globally, almost 1.7 billion people suffer from a disease due to opportunistic fungal species. Immunocompromised people are particularly exposed to very serious infections called invasive fungal infections (IFI). Invasive fungal infections refer to the systemic proliferation of an opportunistic fungus in the host organism to the detriment of the survival of the latter. IFI can affect a variety of organ systems and include conditions such as pulmonary, meninge, sinus, and/or osteo dissemination. The incidence of IFI is increasing, largely because of rising numbers of immunocompromised patients, including those with neutropenia, HIV, chronic immunosuppression, indwelling prostheses, burns and diabetes mellitus, and those taking broad-spectrum antibiotics. Of note, IFIs contribute to substantial morbidity and mortality in immunocompromised patients: despite current therapies, IFIs lead to death for more than half of those infected patients. The main pathogens involved in IFIs belong to Candida, Aspergillus and Cryptococcus species. As of today, there are three main classes of antifungals used in the treatment of invasive fungal infection (IFI), namely polyene antifungals such as amphotericin B, azole antifungals such as fluconazole or voriconazole and echinocandins such as caspofungin.

Among these compounds, amphotericin B is the gold standard for antifungal treatment due to its large spectrum of action and the low incidence of drug resistance. Amphotericin B was shown to be effective in the treatment of fungal infections such as candidiasis, aspergillosis, and cryptococcosis as well as severe tropical fungal diseases such as blastomycosis and coccidioidomycosis. Amphotericin B is also active against protozoan infections such as leishmaniasis. Amphotericin B was isolated from Streptomyces nodosus broth in 1953. Similarly to other antifungal polyenes, Amphotericin B acts by binding ergosterol, a sterol found in fungi and protozoa cell membranes, which depolarizes the membrane and causes the formation of pores resulting incell death. Unfortunately, in spite of its large spectrum and the low resistance incidence, its use in human therapy remains limited to the management of severe, life-threatening infections, especially in immunocompromised patients due to its narrow therapeutic windows. Indeed, amphotericin B is responsible of frequent adverse effects, with nephrotoxicity being the most serious. Nephrotoxicity encompasses well-known tubular damages and can even result in acute renal failure. Amphotericin B-induced nephrotoxicity is not fully understood and certainly multifactorial. It involves the high affinity of amphotericin B to cholesterol, which may result in a high exposure of kidney cells to the drug, due to their high expression in lipoprotein receptors. Several strategies have been developed to improve the solubility and/or decrease the adverse side effects of amphotericin B (AmB).

The original formulation for intravenous injection was based on the complexation of amphotericin B with sodium deoxycholate to improve solubility. Then several liposomal or lipid complex formulations were developed to improve the solubility as well as the tolerability of amphotericin-B:
The lipid complex (ABLC) co-developed by Enzon Pharmaceuticals and Cephalon was marketed under the tradename Abelcet®. Abelcet® consists in AmB complexed with two phospholipids, namely 1-a-dimyristoylphosphatidylcholine (DMPC) and 1-a-dimyristoylphosphatidylglycerol (DMPG).
The colloidal dispersion (ABCD) developed by Three River Pharmaceuticals laboratories and marketed under the names Amphocil® or Amphotec® wherein AmB was complexed with cholesteryl sulfate to form a colloidal dispersion. The drug was discontinued in 2011.
The liposomal preparation (L-AmB) developed by Gilead and Astellas Pharma under the tradename Ambisome®. Ambisome® consists of unilamellar bilayer liposomes made of phosphatidylcholine, cholesterol, and distearoyl phosphatidylglycerol in which AmB is intercalated within the membrane.

These formulations were shown to have less renal toxicity and fewer infusion-related reactions than the original formulation. However, these formulations have a significant high cost of production, which limits their access in low-income countries. In addition, they suffer from other major drawbacks: Abelcet® exhibits a high clearance and provides a lower Cmax than the original drug while Ambisome® have limited diffusion in kidney and lungs.

The improvement of the solubility of AmB by chemical modifications has been also investigated. For instance, Sedlak et al. (Bioorganic & Medicinal Chemistry Letters, 2001, 11, 2833-2835) described the synthesis of AmB-polyethylene glycol (PEG) conjugates. These conjugates were shown to have enhanced solubility in water but a significant decrease in antifungal activity compared to free AmB was observed by bioassays performed in vitro (Tan et al., 2016, PLOS ONE|DOI:10.1371/journal.pone.0152112). Conjugates with other structures such as B-calix[4]arene (Paquet et al., Bioconj. Chem, 2006, 1460-3) and AmB derivatives with double alkylation of the amino group (WO2007096137) have been also described.

However, there is still a need for new derivatives of antifungal drug with improved solubility, improved distribution, better targeting of the infection sites and/or less side effects than the antifungal drug currently on the market.

### SUMMARY OF THE INVENTION

The invention relates to an antifungal prodrug of formula (A):

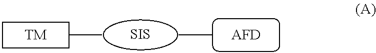

wherein
AFD refers to an antifungal drug,
SIS refers to a self-immolative spacer which is covalently bound to AFD and to TM, and
TM refers to a trigger moiety selected from glycosyl residues and oligosaccharides, said TM stabilizes SIS and is cleavable by a pathogen hydrolytic enzyme which is preferably an extracellular glycosidase (EC 3.2.1), and
   wherein when TM is cleaved by the pathogen hydrolytic enzyme, SIS undergoes a spontaneous degradation so as to release AFD.

In some embodiments, the antifungal prodrug of formula (A) is such that:
TM is selected from the group consisting of hexosamines, N-acetyl hexosamines, neuraminic acid, sialic acid and oligosaccharides thereof comprising from 2 to 50, preferably, from 2 to 10 glycosyl residues and/or
AFD is selected from the group consisting of azole antifungals, polyene antifungals, echinocandins, orotomides and enfumafungin aglycon derivatives.

In some embodiments, TM is selected from the group consisting of glucosamine, galactosamine, mannosamine, neuraminic acid, N-acetylglucosamine, N-acetylgalactosamine, sialic acid, N-acetyl mannosamine and chitine. For instance, TM is N-acetylglucosamine or N-acetylgalactosamine.

In some other embodiments, the AFD is selected from the group consisting of amphotericin B, nystatin, natamycin, caspofungin, micafungin, anidulafungin, rezafungin, votriconazole, ketoconazole, itraconazole, fluconazole, ibrexafungerp, olorofim and derivatives thereof. In preferred embodiments, AFD is caspofungin, votriconazole or amphotericin B, more preferably amphotericin B.

In some embodiments of the prodrug of the invention, SIS is selected from self-immolative spacers which undergo spontaneous degradation involving an electronic cascade or a cyclization. For instance, SIS may comprise or consist in a moiety of formula (Ia1), (Ib1). (Ic1) or (Id1):

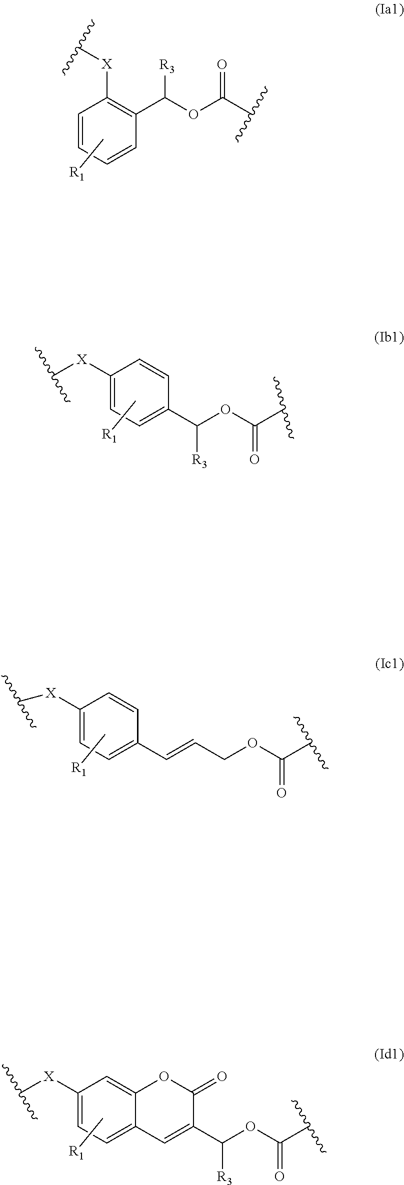

Wherein
X is O, S, —O(C═O)—NH—, O(C═O)O—, —O(P═O)O—, —O(P═S)O—, NR, with R is H or a C₁-C₃ alkyl, preferably CH₃
R₁ is H, a halogen such as F, Br, or Cl, —NO₂, C₁-C₃ alkyl, —CF₃—NHR, —OR, —C(═O)OR, —SO₂R, with R is H or a C₁-C₃ alkyl, preferably CH₃, or a targeting moiety, and
R₃ is H or a targeting moiety, and
R₁ and R₃ are not a targeting moiety at the same time.

Preferably, R₃ is H and R₁ is H, a halogen, —NO₂, —CF₃—OR, —C(═O)OR, —SO₂R, with R is H or a C₁-C₃ alkyl, preferably CH₃.

In a particular embodiment, the antifungal prodrug of formula (A) is selected from compounds of formula (A2):

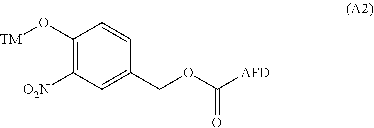

Wherein:
TM is a glycosyl residue selected from the group consisting of glucosamine, galactosamine, N-acetylglucosamine, N-acetylgalactosamine, mannosamine neuraminic acid, and sialic acid.
AFD is an antifungal drug selected from the group consisting of amphotericin B, nystatin, natamycin, caspofungin, micafungin, anidulafungin, rezafungin, votriconazole, ketoconazole, itraconazole, fluconazole and derivatives thereof, preferably from amphotericin B, caspofungin and votriconazole,

and pharmaceutical acceptable salts thereof.

An example of an antifungal prodrug of the invention is:

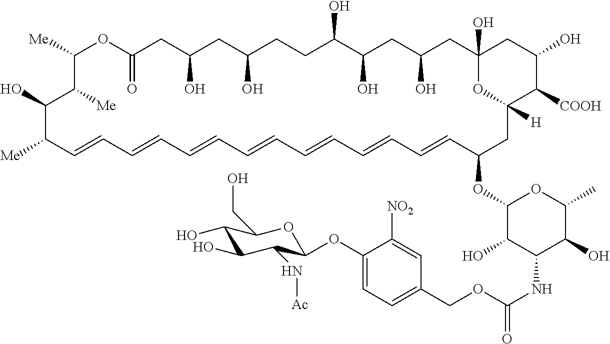

or a pharmaceutically acceptable salt thereof.

The invention also relates to the use of an antifungal prodrug as defined above for the treatment or the prevention of an infectious disease. The infectious disease may be caused by a pathogen belonging to Candida, Aspergillus, Cryptococcus, Mucorales, Fusarium, Scedosporium, Lomentospora, Blastomyces, Mucorales order or Leishmania, Trypanosoma, Plasmodium species. The antifungal prodrug is particularly useful for treating or preventing an invasive fungal disease in an immunocompromised subject.

The invention also relates to a pharmaceutical composition comprising the antifungal prodrug as defined above and a pharmaceutically acceptable excipient.

The invention also relates to a method for treating or preventing an infectious disease in a subject, which comprises administering an effective amount of an antifungal prodrug as defined herein, preferably by oral or intravenous route.

The Invention further relates to the use of an antifungal product as defined herein in the preparation of a pharmaceutical composition for the treatment or the prevention of an infectious disease, preferably for oral or intravenous administration.

### FIGURES

FIG. 1A shows an AmB prodrug of the invention. This compound provided the proof-of-concept of the invention and assessed in the example section of the instant application.

FIG. 1B shows the mechanism of release of AmB from the prodrug which includes the hydrolysis of the target moiety by fungal hydrolytic enzyme followed by the spontaneous decomposition of the self-immolative spacer.

FIG. 2 shows the synthesis pathway and the reaction conditions used to prepare AmB prodrug.

FIG. 3 shows the kinetic of releases of AFD from the AmB prodrug when incubating with a β-N-acetylhexosaminidase as well as the kinetic of formation of intermediate 3 and residue 5 shown in FIG. 1B. Of note, AmB prodrug is stable in aqueous medium at 37° C. (in the absence of the enzyme).

FIG. 4a shows the survival in a mouse model of C. albicans blastoconidia infection for different animal groups, namely (i) treated with Fungizone®, (ii) treated with Ambisome®, (iii) treated with the AmB prodrug of the invention (Compound of FIG. 1A called here GOG) and (iv) administered with vehicle (control.

FIG. 4B shows the fungal charge in kidney determined after euthanasia in a mouse model of C. albicans blastoconidia infection for different groups, namely (i) treated with Fungizone®, (ii) treated with Ambisome®, (iii) treated with the AmB prodrug of the invention (Compound of FIG. 1A called here GOG) and (iv) administered with vehicle (control).

FIG. 5A shows survival curves of G. mellonella treated with amphotericin B (AmB), the AmB prodrug of the invention (Compound of FIG. 1A) and controls.

FIG. 5B shows survival curves of G. mellonella infected with Cryptococcus neoformans treated with amphotericin B (AmB), the AmB prodrug of the invention (Compound of FIG. 1A) and controls.

### DETAILED DESCRIPTION OF THE INVENTION

The Inventors have conceived a new prodrug of amphotericin B having an improved solubility, biodistribution, tolerability and a better targeting of the infection site than amphotericin B. This new prodrug is based on a vectorization platform enabling to increase the solubility, to mask the toxicity of the fungal drug and to promote the release of the active drug at the very precise site of the infection.

This vectorization platform is based on a trigger moiety which is linked to the antifungal drug by a self-immolative group. The trigger moiety stabilizes the self-immolative group and is chosen so as to be selectively recognized and cleaved by hydrolytic enzymes spontaneously secreted by the pathogens at the site of the infection. Following the cleavage of the trigger moiety, the self-immolative group spontaneously undergoes rearrangement leading to the release of the active fungal drug.

Thus, the vectorization platform conceived by the Inventors takes advantage of the fact that pathogens such as fungi spontaneously secrete hydrolytic enzymes in the site of infection. Selecting a trigger moiety which is specific to the hydrolytic enzymes secreted by the pathogens enable to limit the release of the antifungal drug at the very site of fungal infection while preventing damages to the patient's cells and thus limiting side effects.

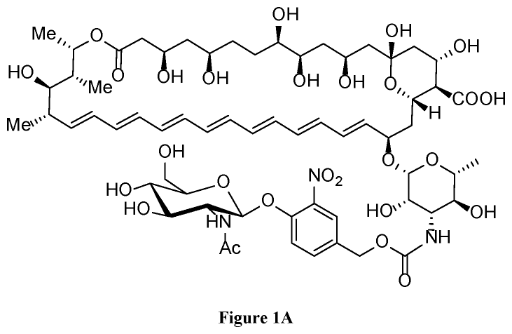

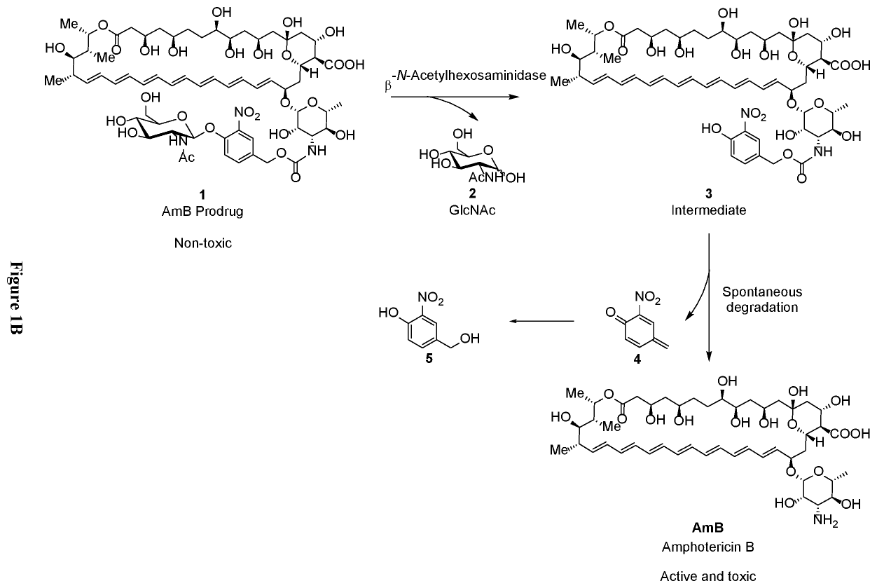

The Inventors provided a proof-of-concept of their innovative vectorization platform with AmB. They conceived an AmB prodrug as shown in FIG. 1A wherein the vectorization platform is linked to the amino group of the mycosamine and comprises N-acetylglucosamine as trigger moiety and 4-hydroxy-3-nitrobenzylic alcohol as self-immolative group.

As illustrated in the Example section, the Inventors demonstrated that AmB is quickly released from the prodrug, upon the action of β-N-acetylhexosaminidase.

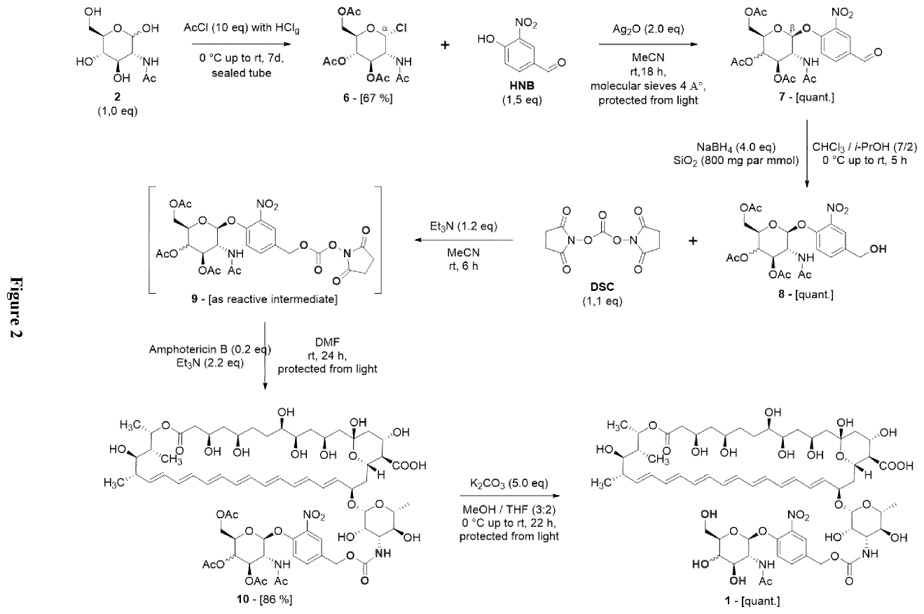

As explained in FIG. 1B and shown in FIG. 2, the hydrolytic enzyme efficiently cleaves the trigger moiety, namely the N-acetylglucosamine group, which results in the release of Intermediate 3. Intermediate 3 spontaneously undergoes a rearrangement to release the active drug AmB.

Of note, it was shown that the prodrug is stable in aqueous buffer at 37° C., without undergoing any significant hydrolysis.

Then, the Inventors assessed the antifungal activity of the prodrug on different fungal cell types, namely blastospores and filamented yeasts as well as Leishmania promastigotes and intracellular amastigotes. Of note, the prodrug was shown to be as effective as AmB on these different cells, which confirms that AmB is effectively released from the prodrug by the action of pathogen hydrolytic enzymes.

Moreover the prodrug does not exhibit any significant toxicity on HELA cells in contrast to AmB which has an IC50 of about 23 μM. Thus, the prodrug is not metabolized by, and does not have any significant toxicity with respect to, human cells.

The Inventors also showed that the AmB prodrug of the invention was at least as effective as Fungizone® (AmB) and Ambisome® (AmB in liposomal formulation) to treat fungal infection in a mouse model of C. albicans blastoconidia infection. Of note, the group treated with the AmB prodrug of the invention showed a significant improvement in survival and a significant decrease in the kidney fungal load as compared to the control group.

The Inventors also studied the effects of AmB and AmB prodrug of the invention on Galleria mellonella model, a larval model enabling to assess the efficacy and the intrinsic toxicity of active drugs. The Inventors showed that the AmB prodrug of the invention is significantly less toxic than AmB, confirming the data obtained on human cell lines.

Besides, the AmB prodrug was shown to be effective against Cryptococcus neoformans and Cr gatti infection in the Galleria melonella model in the same order of magnitude as AmB. All together, these results strongly support the fact that the vectorization platform conceived by the Inventors does not impair the antifungal activity of the drug, increases its solubility and promotes its specific release near the site of infection, while preventing adverse side effects caused by the large diffusion of the antifungal. The therapeutic window of antifungal drug is therefore increased.

Without to be bound by any theory, the Inventors consider that this vectorization platform used to vectorize AmB can be also effective for the vectorization of other antifungal drugs such as echinocandins.

Accordingly, the invention relates to an antifungal prodrug of formula (A):

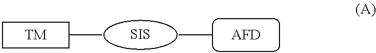

Wherein
AFD refers to an antifungal drug,
SIS refers to a self-immolative spacer which is covalently bound to AFD and to TM, and
TM refers to a trigger moiety which stabilizes SIS and can be cleaved by a pathogen hydrolytic enzyme,

Wherein when TM is cleaved, SIS undergoes a spontaneous degradation so as to release AFD. Thus, the active AFD is released from the prodrug of the invention via a two steps process including (i) the enzymatic hydrolysis of the covalent bond between TM and SIS and (ii) the spontaneous decomposition of SIS.

The Antifungal Drug (AFD)

As used herein, an antifungal drug (AFD) refers to any drug having a fungicide or fungistatic activity on at least one pathogenic fungal species. In some embodiments, the antifungal drug is active on at least one pathogenic fungus belonging to Candida, Aspergillus and Cryptococcus species. In a particular embodiment, the antifungal drug has a broad spectrum activity, which means that it exhibits an antifungal activity against a plurality of fungal species.

The antifungal drug typically has a molecular weight of less than 2 000 g·mol⁻¹, preferably of less than 1 500 g·mol⁻¹.

Antifungal drugs encompass, without to be limited to, azole antifungals, polyene antifungals, echinocandins, orotomides and enfumafungin aglycon derivatives.

As used herein, azole antifungals refer to antifungal compounds comprising at least one five-membered heterocyclic moiety which contains a nitrogen atom and at least one other non-carbon atom (i.e. nitrogen, sulfur, or oxygen) as part of the ring. Preferred heterocycles are triazole and imidazole. Azole antifungals may act by blocking the conversion of lanosterol to ergosterol by inhibition of lanosterol 14α-demethylase. Azole antifungals, encompass, without being limited to, ketoconazole, itraconazole, fluconazole, efinaconazole, albaconazole, voriconazole, ravuconazole, and posaconazole.

The azole antifungal may be linked to the self-immolative spacer (SIS) e.g. through its hydroxyl group when present.

As used herein, polyene antifungals (also called herein polyene antibiotics or polyene antimycotics) refer to antimycotic drugs which comprise a macrocycle containing a heavily hydroxylated region opposite to a region comprising a plurality of conjugated double bonds (polyene moiety). The macrocycle of polyene antifungals generally bears an aminoglycoside such as D-mycosamine.

Polyene antifungal drugs generally act as ionophores. They bind to ergosterol, a major component of the fungal cell membrane and form pores in the membrane that lead to K+ leakage, acidification, and death of the fungus.

Polyene antifungals encompass, without being limited to, amphotericin A and B, nystatin, and natamycin, rezafungin, rimocidin, filipin, hamycin, and perimycin.

When the antifungal drug (AFD) is a polyene antifungal comprising an aminoglycoside group, said AFD is preferably linked to the self-immolative spacer (SIS) through the amino group of said aminoglycoside. Otherwise, the AFD may be linked to SIS through one of the hydroxyl groups present on the macrocycle.

As used herein, echinocandins refer to macrocyclic lipopeptide antifungal drugs which works by inhibiting the enzyme (1→3)-β-D-glucan synthase and thereby disturbing the integrity of the fungal cell wall. The structure of echinocandins typically comprises a lipophilic tail linked to a peptidic macrocycle. Echinocandins encompass without being limited to caspofungin, micafungin, anidulafungin, rezafungin, echinocandin B (also known as CD 101—CAS N° 1396640-59-7), pneumocandin B₀, biafungin, and aminocandin. When the AFD is an echinocandin, it may be linked to the SIS through one of its free hydroxyl or amino group, preferably through one of its primary amino group if present.

As an alternative to echinocandins, one can use enfumafungin aglycon derivatives such as Ibrexafungerp (also known as SCV 078 and MK 3118). Similarly to echinocandins, these compounds are inhibitors of fungal beta-1,3-D-glucan synthases. Ibrexafungerp is a new antifungal drug under development (phase III clinical trial on going). Its CAS number is 1207753-03-04. Other enfumafungin aglycon derivatives of interest are disclosed in patent application WO2010019203.

As used herein, orotomides refer to a new class of antifungals comprising pyrrole moiety and acting by stopping pyrimidine biosynthesis in fungal cells. Orotomides cause reversible inhibition of dihydroorotate dehydrogenase (DHODH). This inhibition in turn block the growth of hyphae.

Orotomides of interest are for instance described in patent application WO2016079536. A preferred orotomide is orofim (CAS N° 1928707-56-5) which is currently under phase III clinical trial.

As used herein, “a derivative” refers to any AFD comprising one or several chemical modifications while keeping its antifungal activities.

In some embodiments, the AFD is selected from the group consisting of amphotericin B, nystatin, natamycin, caspofungin, micafungin, anidulafungin, rezafungin, votriconazole, ketoconazole, itraconazole, fluconazole, ibrexafungerp, olorofim and derivatives thereof.

The Self-Immolative Spacer (SIS)

The self-immolative spacer (SIS) (also called herein self-immolative group) is a chemical group which links the antifungal drug (AFD) and the Trigger Moiety (TM) together and which undergoes spontaneous decomposition once TM is cleaved.

Indeed, when the trigger moiety (TM) is released, i.e. when the covalent bond between TM and SIS is cleaved by the action of a pathogen hydrolytic enzyme, SIS spontaneously undergoes a structural rearrangement leading to the release of the active antifungal drug in the site of the infection.

SIS is selected so as to increase the solubility of AFD and/or limit the steric hindrance around TM which enable the recognition of TM by the hydrolytic enzyme of interest. SIS is also selected so as to rapidly decompose once TM is cleaved by the fungal hydrolytic enzyme, whereby AFD is released.

SIS may be a bifunctional spacer or a trifunctional spacer. When a trifunctional SIS is used, SIS bears a further entity, e.g. an additional AFD moiety, a moiety for increasing solubility such as PEG moiety, or a targeting moiety, as defined further below.

Self-immolative groups are well-known in the state in the art and have been extensively studied. One can refer to Schmidt et al., Angew. Chem. Int, 2015, 54, 7492-7509 which is a review about self-immolative spacers, the content of which being incorporated within by reference. As explained by Schmidt et al., the spontaneous decomposition of self-immolative group is mainly driven by two types of processes namely (i) electronic cascade which may lead to the formation of a quinone or azaquinone and (ii) cyclization which may lead to imidazolidinone, oxazolidinone or 1,3-oxathiolan-2-one ring structures.

In some embodiments, the self-immolative spacer relies on an electronic cascade for disassembly and comprises an aromatic structure bearing O-, N- or S-group.

In a particular embodiment, SIS comprises, or consists in, a moiety of formula (Ia), (Ib), (Ic) or (Id):

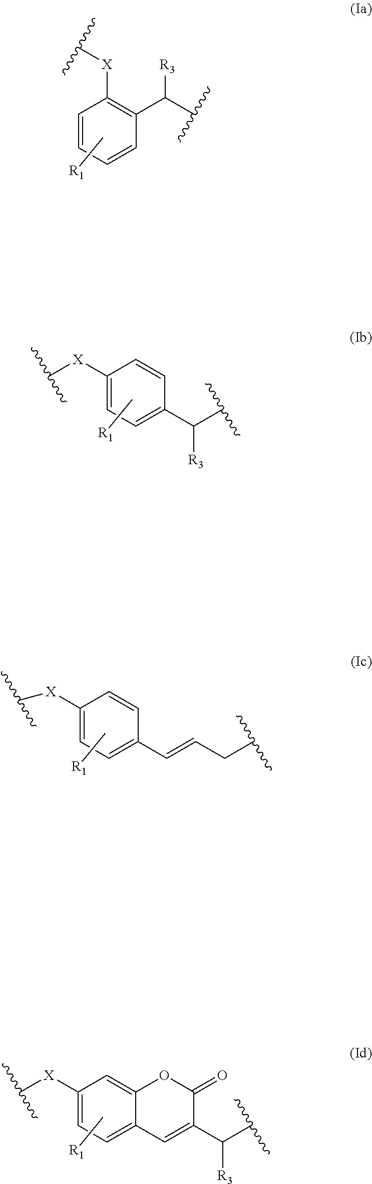

Wherein
X is O, S, —O(C═O)—NH—, O(C═O)O—, —O(P═O)O—, —O(P═S)O—, NR, with R is H or a C₁-C₃ alkyl, preferably CH₃
R₁ is H, a halogen such as F, Br, or Cl, —NO₂, C₁-C₃ alkyl, —CF₃—NHR, —OR, —C(═O)OR, —SO₂R, with R is H or a C₁-C₃ alkyl, preferably CH₃, or a targeting moiety, and
R₃ is H or a targeting moiety.

R₁ may be at position para, meta or ortho of the X group. Preferably, R₁ is at position ortho or para. Preferably, R₁ and R₃ are not a targeting moiety at the same time.

As used herein, “a targeting moiety” refers to any group enabling the delivery of the prodrug to a specific organ, tissue or cell of the subject, or to a specific pathogen. The targeting moiety may be of any types. Typically, the targeting moiety is able to specifically bind to a target component expressed by the organ, tissue, cell or pathogen to target.

For instance, the targeting moiety may be selected from antibodies, a fragment or derivative of an antibody such as Fab, Fab′, and ScFv, an aptamer, a spiegelmer, a peptide aptamer, and a ligand or a substrate of the target component of interest. Said ligand or substrate can be of any type such as small chemical molecules having a molecular weight of less than 1000 g·mol⁻¹, peptides, sugars, hormones, oligosaccharides, proteins, and a receptor or receptor fragment able to bind to the target component.

The targeted component may be, for instance, a membrane protein, such as a membrane receptor, a membrane or cell wall components, and the like. In a particular embodiment, the targeted component is a component of the pathogen cell wall or a component present on the surface of the pathogen such as Asl3 (Agglutinin-like protein 3), HWP1 (Hyphal Protein 1), beta-D-glucan or the external fragment of HSP90 (heat shock protein 90).

Accordingly, the target may be Asl3, HWP1, HSP90, or beta-D-glucan. In certain embodiments, the targeting moieties comprise a spacer enabling its covalent binding with the core structure of SIS while limiting the steric hindrance and/or increasing the solubility. For instance, the spacer may be a hydrophilic one such as PEG-based spacer.

In a particular embodiment, SIS is a moiety of formula (Ia1), (Ib1), (Ic1) or (Ia1):

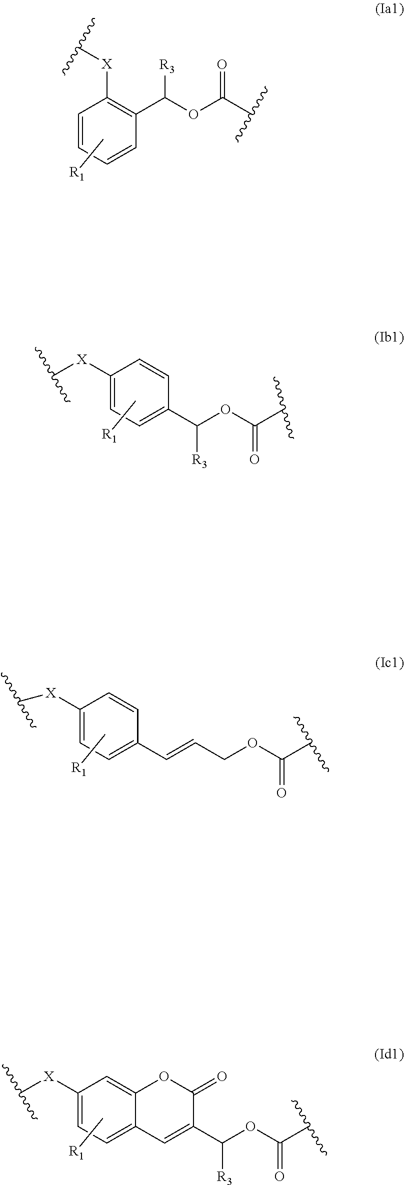

Wherein X, R₁ and R₃ are as defined above.

In a particular embodiment, the SIS comprises, or consists of formula (Ib2):

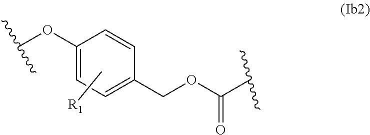

Wherein R₁ is as defined above. In a preferred embodiment, R₁ is selected from the group consisting of H, a halogen such as F, Br, or Cl, —NO₂, —CF₃, —C(═O)OR, and —SO₂R, with R is H or a C₁-C₃ alkyl, preferably CH₃. R₁ may be at position ortho or para, preferably at position ortho.

In some other embodiments, the self-immolative spacer relies on cyclization mechanism and comprises an alkyl chain and/or an aromatic moiety. For instance, the self-immolative spacers may comprise, or may consist of, a moiety of formula (Ie), (If), (Ig), (Ih) or (Ii)

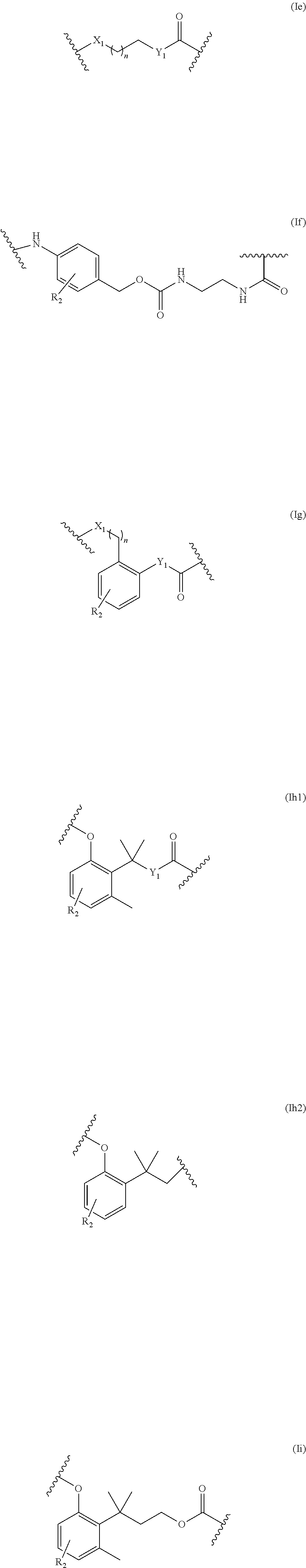

Wherein:
X₁ is CH₂, O, S, NR with R is H or a C₁-C₃ alkyl, preferably CH₃,
Y₁ is CH₂, O, NH, or a single bond
R₂ is H, a halogen such as F, Br, or Cl, —NO₂, C₁-C₃ alkyl, —CF₃—NHR, —OR, —C(═O)OR,
   —SO₂R, with R is H or a C₁-C₃ alkyl, preferably CH₃, or a targeting moiety, and
n is an integer from 1 to 5, preferably 1 or 2.

As mentioned above, the prodrug may comprise a targeting moiety which is typically borne by the self-immolative spacer. The self-immolative spacer may be a trifunctional linker which binds together TM, AFD and the targeting moiety. Such self-immolative spacers, also called chemical adaptors, are described for instance in Gopin et al. Bioorg. Med. Chem. 2004, 12, 1853-1858 and in Gopin et al. Angew. Chem. Int. Ed. 2003, 42, 327-332.

For instance, SIS may comprise, or consist in, a moiety of formula (Ij) or (Ik):

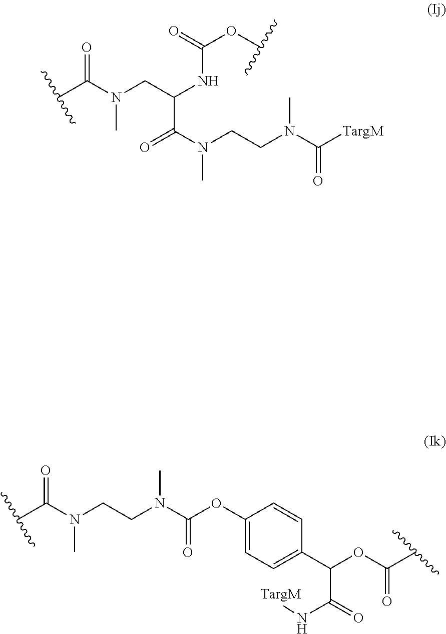

Wherein TargM is a targeting moiety as defined above.

In a particular embodiment, SIS comprises or consists of a moiety of formula (Ib3):

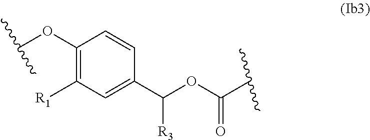

Wherein R₁ is as defined above, and R₃ is H or a targeting moiety (TargM).

Preferably, R₁ is selected from the group consisting of H, a halogen such as F, Br, or Cl, —NO₂, —CF₃, —C(═O)OR, and —SO₂R, with R is H or a C₁-C₃ alkyl, preferably CH₃

Accordingly, the prodrug of the invention may be of formula (A1):

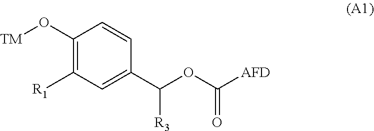

With R₁ is as defined above and R₃ being H or a targeting moiety, preferably H.

In a particular embodiment, R₃ is H and R₁ is NO₂. The prodrug is thus of formula (A2):

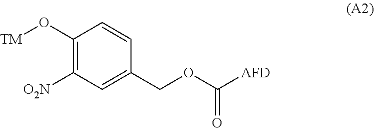

The Trigger Moiety (TM)

As used herein, the trigger moiety (TM) refers to a chemical group which stabilizes SIS, i.e. prevents its spontaneous decomposition and thus acts as a protective group. In the context of the invention, TM is selected so as to be selectively cleaved by the action of an enzyme expressed by a pathogen of interest. Enzymes of interest are pathogen hydrolytic enzymes, e.g. fungal hydrolytic enzymes, secreted in the extracellular environment and able to catalyze the release of a glycosyl moiety from a substrate of interest.

For instance, the pathogen hydrolytic enzyme is an extracellular glycosidase (EC 3.2.1) able to catalyze the hydrolysis of O-, N- or S-glycosides. The hydrolytic enzymes of interest encompass, without being limited to, beta-N-acetylhexosaminidases (EC 3.2.1.52), beta-N-acetylgalactosaminidase (EC 3.2.1.53), chitinase (EC 3.2.1.14), beta-glucosidase (EC 3.2.1.21), alpha-D-mannosidase (EC 3.2.1.24), beta-D-mannosidase (EC 3.2.1.25), chitobiase (EC 3.2.1.29), beta-D-acetylglucosaminidase (EC 3.2.1.30), exo-alpha-sialidase (EC 3.2.1.18), endo-alpha-sialidase (EC 3.2.1.129), exo-1,4-β-D-glucosaminidase (EC 3.2.1.165).

Accordingly, the trigger moiety is typically a glycosyl group. In some embodiments of the invention, the trigger moiety is selected from hexosamines and N-acetyl hexosamines, preferably from N-acetyl hexosamines. The trigger may be also selected from 9-carbon sugars such as neuraminic acid and sialic acid such as N-acetylneuraminic acid.

The trigger moiety may be also selected from oligosaccharides based on hexosamines or and N-acetyl hexosamines, such as chitine, and/or based on neuraminic or sialic acid. Typically the oligosaccharides may comprise from 2 to 50 glycosyl residues, such as from 2 to 10 glycosyl residues.

As used herein, hexosamines refer to hexoses wherein one of the hydroxyl groups has been replaced by an amino group. Hexosamines encompass, without being limited to, fructosamine, galactosamine, glucosamine, and mannosamine.

In some embodiments, the trigger moiety (TM) is selected from the group consisting of glucosamine, galactosamine, mannosamine, N-acetylglucosamine, N-acetylgalactosamine, N-acetyl mannosamine, chitine neuraminic acid and sialic acid.

In an additional embodiment, TM is selected from N-acetylglucosamine, N-acetylgalactosamine, N-acetyl mannosamine, and sialic acid moieties. For instance, the trigger moiety is N-acetylglucosamine or N-acetylgalactosamine. Such glycosyl residues may be cleaved by fungal beta-N-acetylhexosaminidases (EC 3.2.1.52).

Examples of Compounds According to the Invention

In some embodiments, the prodrug of the invention is of formula (A):

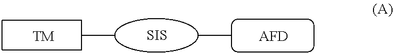

Wherein
TM is a glycosyl residue selected from the group consisting hexosamines, N-acetylhexosamines, neuraminic acid, sialic acid and oligosaccharides thereof comprising from 2 to 50, preferably from 2 to 10 glycosyl residues,
SIS is a self-immolative spacer comprising, or consisting in, a moiety of formula (Ia), (Ib), (Ic), (Id), (Ij), or (Ik), and
AFD is an antifungal drug selected from azole antifungals, polyene antifungals, echinocandins, orotomides, enfumafungin aglycon derivatives and derivatives thereof,

or a pharmaceutically acceptable salt thereof.

In some other embodiments, the prodrug of the invention is of formula (A) wherein:
TM is a glycosyl residue selected from the group consisting of glucosamine, galactosamine, mannosamine, N-acetylglucosamine, N-acetylgalactosamine, N-acetyl mannosamine residues, neuraminic acid, sialic acid and chitine,
SIS is self-immolative spacer comprising, or consisting in, a moiety of formula (Ia1), (Ib1), (Ic1), (Id1), (Ib2) or (Ib3), and
AFD is an antifungal drug selected from amphotericin B, nystatin, natamycin, caspofungin, micafungin, anidulafungin, rezafungin and derivatives thereof.

In some embodiments, the prodrug is of formula (A) wherein:
TM is a glycosyl residue selected from the group consisting of glucosamine, galactosamine, mannosamine, N-acetylglucosamine, N-acetylgalactosamine, and N-acetyl mannosamine residues,
SIS is a self-immolative spacer,
AFD is an antifungal drug selected from amphotericin B, caspofungin, and derivatives thereof.

SIS may comprise, or consist in, a moiety of any one of formula (Ia), (Ib), (Ic), (Id), (Ie), (If), (Ig), (Ih), (Ij), or (Ik).

In some other embodiments, the prodrug of the invention is of formula (A) wherein:
TM is N-acetylglucosamine residue and N-acetylgalactosamine residue, preferably N-acetylgalactosamine residue,
SIS is a self-immolative spacer,
AFD is an antifungal drug selected from amphotericin B, caspofungin, and derivatives thereof.

SIS may comprise, or consist in, a moiety of any one of formula (Ia), (Ib), (Ic), (Id), (Ie), (If), (Ig), (Ih1), (Ih2), (Ij), (Ik), (Ib2) and (Ib3), preferably any one of formula (Ia1), (Ib1), (Ic1), (Id1), (Ib2) and (Ib3).

In some preferred embodiments, AFD is amphotericin B.

In other embodiments, the prodrug of the invention is of formula (A1) wherein:

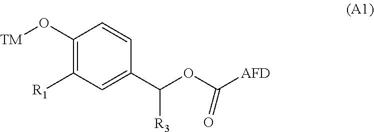

Wherein
R₁ is H, a halogen such as F, Br, or Cl, —NO₂, C₁-C₃ alkyl, —CF₃—NHR, —OR, —C(═O)OR, —SO₂R, with R is H or a C₁-C₃ alkyl such as CH₃,
R₃ is H or a targeting moiety, preferably H.
TM is a glycosyl residue selected from the group consisting of glucosamine, galactosamine, mannosamine, N-acetylglucosamine, N-acetylgalactosamine, and N-acetyl mannosamine, and
AFD is an antifungal drug selected from polyene antifungals and echinocandins such as amphotericin B, nystatin, natamycin, caspofungin, micafungin, anidulafungin, rezafungin, votriconazole, ibrexafungerp, olorofim and derivatives thereof, or a pharmaceutically acceptable salt thereof.

In an additional embodiment, the prodrug of the invention is of formula (A1) wherein:
R₁ is H, a halogen such as F, Br, or Cl, —NO₂, —CF₃, —NHR, —C(═O)OR, —SO₂R, with R is H or a C₁-C₃ alkyl such as CH₃,
R₃ is H
TM is a glycosyl residue selected from the group consisting of glucosamine, galactosamine, N-acetylglucosamine, and N-acetylgalactosamine,
AFD is an antifungal drug selected from polyene antifungals, echinocandins, orotomides and enfumafungin aglycon derivatives such as amphotericin B, nystatin, natamycin, caspofungin, micafungin, anidulafungin, rezafungin, votriconazole, ibrexafungerp, olorofim and derivatives thereof or a pharmaceutically acceptable salt thereof.

In a further embodiment, the prodrug of the invention is of formula (A2)

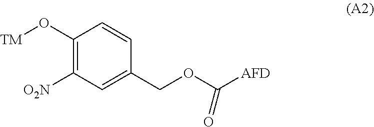

Wherein:
TM is a glycosyl residue selected from the group consisting of glucosamine, galactosamine, N-acetylglucosamine, and N-acetylgalactosamine,
AFD is an antifungal drug selected from polyene antifungals, echinocandins, azole antifungals, orotomides and enfumafungin aglycon derivatives such as amphotericin B, nystatin, natamycin, caspofungin, micafungin, anidulafungin, rezafungin, votriconazole, ketoconazole, itraconazole, fluconazole, Ibrexafungerp, Olorofim and derivatives thereof or a pharmaceutically acceptable salt thereof.

In another embodiment, the prodrug of the invention is of formula (A3):

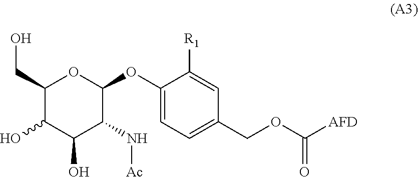

Wherein R₁ is selected from the group consisting of H, —NO₂, —COOMe, preferably —NO₂ and AFD is selected from polyene antifungals, echinocandins, azole antifungals and derivatives thereof, preferably in the group consisting of amphotericin B, nystatin, natamycin, caspofungin, micafungin, anidulafungin, rezafungin, votriconazole and derivatives thereof,
or a pharmaceutically acceptable salt thereof.

Preferred AFD is votriconazole, amphotericin B and caspofungin, more preferably amphotericin B.

For instance, the prodrug of the invention may be one of the following compounds or a pharmaceutically salt thereof:

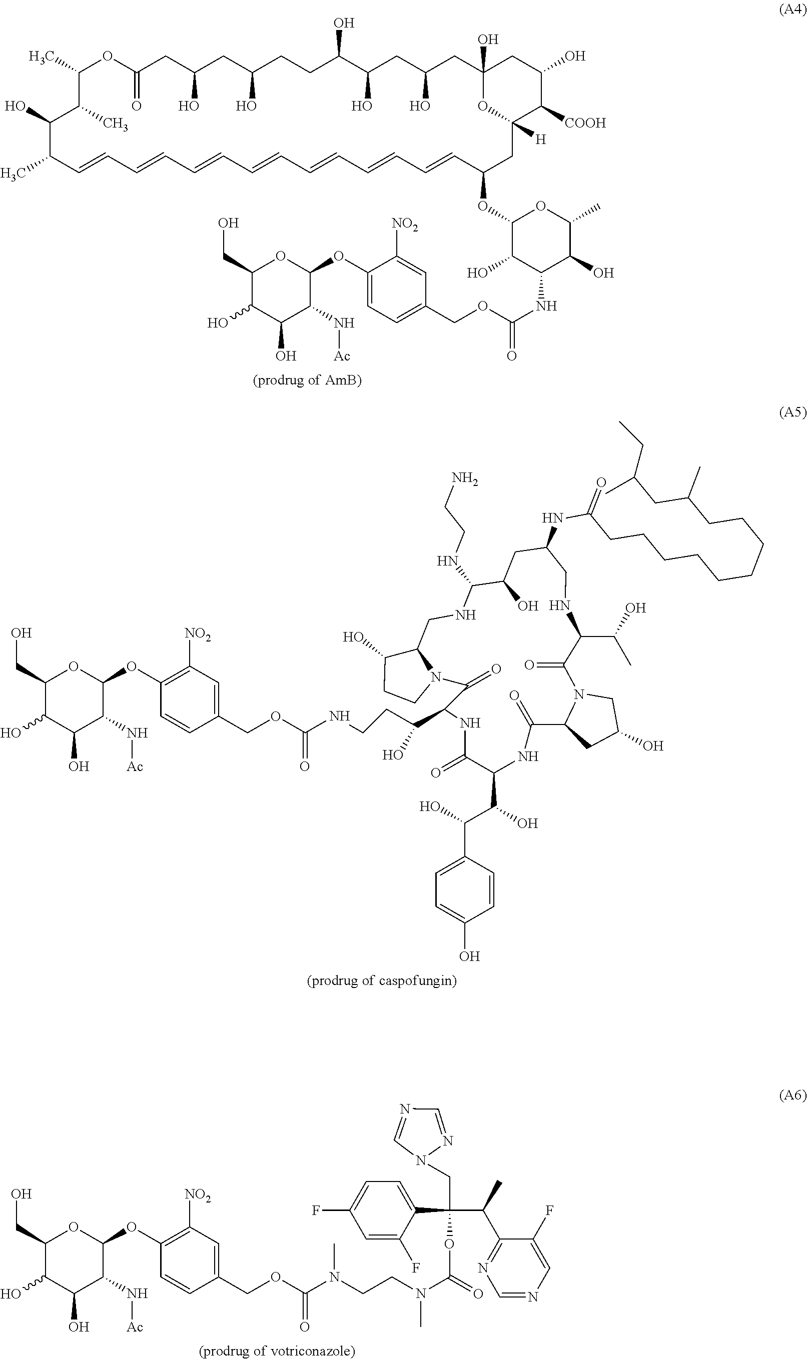

As used herein, the term “pharmaceutically acceptable salt” refers to non-toxic salts, which can generally be prepared by contacting the prodrug of interest (e.g. AmB prodrug) with a suitable organic or inorganic acid. For instance, pharmaceutical salts may be, without being limited to, acetate, benzenesulfonate, benzonate, bicarbonate, bisulfate, bitartrate, bromide, butyrate, carbonate, chloride, citrate, diphosphate, fumarate, iodide, lactate, laurate, malate, maleate, mandelate, mesylate, oleate, oxalate, palmitate, phosphate, propionate, succinate, sulfate, tartrate, and the like.

The prodrugs of the invention can be prepared by standard chemical process. The Example section describes the synthesis of a specific prodrug of the invention, which can be adapted to obtain other prodrugs of interest.

Therapeutic Uses and Methods of the Invention

The invention also relates to the use of a prodrug as defined above in the treatment or the prevention of an infectious disease. An additional object of the invention is a method for treating or preventing an infectious disease in a subject, comprising administering an effective amount of the prodrug of the invention to the subject. The invention also relates to the use of a prodrug of the invention for treating or preventing an infectious disease in a subject.

As used herein, an “infectious disease” (also called herein infection) refers to any disease or disorder, and symptoms thereof, caused or resulted from the contamination of the subject by a pathogen, such as a pathogenic bacterium, fungus including yeast and mold, or protozoa, or a virus. In preferred embodiments, the infectious disease is caused by a pathogenic fungus e.g. a pathogenic yeast or mold or by a pathogenic protozoan, more preferably a pathogenic fungus.

For instance, the infectious disease may be caused by a pathogen belonging to Candida, Aspergillus, Cryptococcus, Mucorales, Fusarium, Scedosporium, Lomentospora, Blastomyces or Leishmania, Trypanosoma, Plasmodium species.

Examples of pathogens include Aspergillus fumigatus, Aspergillus flavus, Candida albicans including C. albicans blastoconidia, Candida krusei, Candida lusitaniae, Candida parapsilosis, Candida tropicalis, Candida glabrata, Candida auris, Cryptococcus neoformans, Cryptococcus gattii, and Blastomyces dermatitidis.

The infectious disease may be systemic, may concern one or several organs, e.g. an organ system such as respiratory tract or gastrointestinal tract or may be local, i.e. localized to a specific organ or tissue such as brain, skin or oral cavity. The infection disease can be an infection of mucosal membranes such as oral, esophageal or vaginal infections, or an infection affected the bone, the skin, the blood, the urogenital tract or the central nervous system of the subject, this list being not exhaustive.

The infectious disease encompasses, without being limited to Candida, Aspergillus cryptococcal infections, mucormycosis infections, blastomycosis, fusariosis, leishmaniasis and the like.

In some embodiments, the infectious disease may be a hospital-acquired infection, i.e. a nosocomial infection or a community-acquired disease.

In some embodiments, the infectious disease is an invasive fungal disease (IFD).

The subject treated with the prodrug of the invention is preferably a mammal, more preferably a human being. The subject may be of any gender and of any age, including neonates, infants, children and aged subjects.

In some embodiments, the subject is immunocompromised. The immunocompromised status of the patient may be a primary immunodeficiency (i.e. caused by congenital or inherited defects) or a secondary immunodeficiency, e.g. resulting from a surgery or from an immunosuppressive treatment such as chemotherapy and anti-rejection drugs, cancers such as leukemia, pathogens such as human immunodeficiency virus (HIV) which causes AIDS, and autoimmune diseases. In certain embodiments, the subject may suffer from a disease which makes him susceptible to infectious diseases. For instance, the patient may be diabetic.

In some other embodiments, the patient has undergone or will undergo a surgery. In such a case, the prodrug of the invention may be used to prevent the onset of the infectious disease in the subject who has undergone or will undergo a surgery. The prodrug of the invention may be also used in order to prevent an infectious disease as described above in a subject who is exposed to the pathogen. For instance, the subject may be a medical staff.

In a particular aspect, the prodrug of the invention may be administered to the subject in combination with an additional therapeutic agent. The administration of the additional therapeutic compound may be simultaneous, separate or successive to the administration of the prodrug of the invention.

As used herein, a “therapeutically effective amount” refers to an amount of the prodrug which prevents, removes, slows down the infectious disease or reduces or delays one or several symptoms or disorders caused by or associated with the said infectious disease in the subject, preferably a human.

The effective amount, and more generally the dosage regimen, of the prodrug of the invention and pharmaceutical compositions thereof may be easily determined and adapted by the one skilled in the art. An effective dose can be determined by the use of conventional techniques and by observing results obtained under analogous circumstances. The therapeutically effective dose of the prodrug of the invention will vary depending on the infectious disease to be treated or prevented, the gravity of the infectious to be treated, the route of administration, any co-therapy involved, the patient's age, weight, general medical condition, medical history, etc. Typically, the amount of the prodrug to be administrated to a patient may range from about 0.001 mg/day/kg to 100 mg/day/kg of body weight, preferably from 0.1 mg/day/kg to 25 mg/day/kg of body weight, more preferably from 0.1 mg/day/kg to 10 mg/day/kg of body weight.

The prodrug of the invention may be administered at least one time a day during several consecutive days, weeks or months until the achievement of the desired therapeutic effect.

The administration of the prodrug of the invention may be topical, parenteral or enteral. Indeed, the prodrug of the invention may be administered by any conventional route including, but not limited to, oral, buccal, sublingual, rectal, intravenous, intra-muscular, subcutaneous, intra-osseous, dermal, transdermal, mucosal, transmucosal, intra-articular, intra-cardiac, intra-cerebral, intra-peritoneal, intranasal, pulmonary, intraocular, vaginal, or transdermal route. Indeed, the administration route of the prodrug of the invention may vary depending on the infectious disease to treat and the organ or tissue of the patient afflicted by the disease.

In some preferred embodiments, the prodrug of the invention is administered by intravenous route or by oral route.

Pharmaceutical Compositions of the Invention

In an additional aspect, the invention relates to a pharmaceutical composition comprising

(i) a prodrug of any one of formula (A), (A1), (A2) or (A3) and described above (or a pharmaceutically acceptable salt or solvate thereof) as an active principle and (ii) at least one pharmaceutically acceptable excipient.

The pharmaceutical composition of the invention may comprise:
from 0.01% to 90% by weight of a prodrug of the invention, and
from 10% to 99.99% by weight of excipients,
   the percentage being expressed as compared to the total weight of the composition.

Preferably, the pharmaceutical composition may comprise:
from 0.1% to 50% by weight of a prodrug of the invention, and
from 50% to 99.9% by weight of excipients.

Such a pharmaceutical composition is preferably to be used in the treatment or the prevention of an infectious disease caused by a fungus such as Candida, Aspergillus and Cryptococcus species or a protozoa such as Leishmania.

The pharmaceutical composition of the invention may be formulated according to standard methods such as those described in Remington: The Science and Practice of Pharmacy (Lippincott Williams & Wilkins; Twenty first Edition, 2005).

Pharmaceutically acceptable excipients that may be used are, in particular, described in the Handbook of Pharmaceuticals Excipients, American Pharmaceutical Association (Pharmaceutical Press; 6th revised edition, 2009). Typically, the pharmaceutical composition of the invention may be obtained by admixing a prodrug of the invention with at least one pharmaceutically excipient.

Examples of appropriate excipients include, but are not limited to, solvents such as water or water/ethanol mixtures, fillers, carriers, diluents, binders, anti-caking agents, plasticizers, disintegrants, lubricants, flavors, buffering agents, stabilizers, colorants, dyes, anti-oxidants, anti-adherents, softeners, preservatives, surfactants, wax, emulsifiers, wetting agents, and glidants. Examples of diluents include, without being limited to, microcrystalline cellulose, starch, modified starch, dibasic calcium phosphate dihydrate, calcium sulfate trihydrate, calcium sulfate dihydrate, calcium carbonate, mono- or disaccharides such as lactose, dextrose, sucrose, mannitol, galactose and sorbitol, xylitol and combinations thereof. Examples of binders include, without being limited to, starches, e.g., potato starch, wheat starch, corn starch; gums, such as gum tragacanth, acacia gum and gelatin; hydroxypropyl cellulose, hydroxyethyl cellulose, hydroxypropyl methyl cellulose; polyvinyl pyrrolidone, copovidone, polyethylene glycol and combinations thereof. Examples of lubricants include, without being limited to, fatty acids and derivatives thereof such as calcium stearate, glyceryl monostearate, glyceryle palmitostearate magnesium stearate, zinc stearate, or stearic acid, or polyalkyleneglycols such as PEG. The glidant may be selected among colloidal silica, dioxide silicon, talc and the like. Examples of disintegrants encompass, without being limited to, crospovidone, croscarmellose salts such as sodium croscarmellose, starches and derivatives thereof. Examples of surfactants encompass, without being limited to, simethicone, triethanolamine, les polysorbate and derivatives thereof such as Tween® 20 or Tween® 40, poloxamers, fatty alcohol such as laurylic alcohol, cetylic alcohol and alkylsulfate such as sodium dodecylsulfate (SDS). Examples of emulsifiers, encompass for example, ethyl alcohol, isopropyl alcohol, ethyl carbonate, ethyl acetate, benzyl alcohol, benzyl benzoate, propyleneglycol, 1,3-butyleneglycol, dimethylformamide, oils, polyethyleneglycol and fatty acid esters of sorbitan or mixtures of these substances.

It goes without saying that the excipient(s) to be combined with the prodrug of the invention may vary upon (i) the physico-chemical properties including the stability of the said active prodrug, (ii) the pharmacokinetic profile desired for said active ingredient, (iii) the dosage form and (iv) the route of administration.

The pharmaceutical composition may be of any type. For instance, the pharmaceutical composition may be a solid oral dosage form, a liquid oral dosage form, a suspension, for instance for intravenous route, a dosage form for topical application such as cream, ointment, gel and the like, a patch, such as a transdermal patch, a muco-adhesive patch or tablet, in particular adhesive plaster or bandage, a suppository, an aerosol for intranasal or pulmonary administration. The pharmaceutical composition may provide an immediate-release, a controlled-release or a prolonged-release of the prodrug of the invention. Oral solid dosage forms encompass, without being limited to, tablets, capsules, pills, and granules. Optionally, said oral solid forms may be prepared with coatings and shells, such as enteric coatings or other suitable coatings or shells. Several such coatings and/or shells are well known in the art. Examples of coating compositions which can be used are polymeric substances and waxes. The prodrug can also be used in microencapsulated form, if appropriate, with one or more of the above-mentioned excipients. Liquid dosage forms for oral administration include pharmaceutically acceptable emulsions, solutions, suspensions, syrups, and elixirs. The liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, polyethyleneglycol and fatty acid esters of sorbitan or mixtures of these substances, and the like. If desired, the composition can also include adjuvants, such as wetting agents, emulsifying and suspending agents, sweetening, flavoring and/or perfuming agents. Suspensions, may contain suspending agents, such as, ethoxylated isostearyl alcohols, polyoxyethylene sorbitol and sorbitan esters, microcrystalline cellulose, aluminum metahydroxide, bentonite, agar-agar, and the like.

Vaginal or rectal suppositories may be prepared by mixing the prodrug of the present invention with suitable non-irritating excipients or carriers such as cocoa butter, polyethyleneglycol, or a suppository wax which are solid at ordinary temperatures but liquid at body temperature.

The ointments, pastes, creams and gels may contain excipients such as oils, waxes, paraffins, starch, tragacanth, cellulose derivatives, polyethylene glycols, silicones, bentonites, silicic acid, talc and zinc oxide, or mixtures thereof. The pharmaceutical composition may be also in the form of aerosol which may be delivered in the lungs by using an inhaler system. For instance the prodrug of the invention may be adsorbed on the surface of nano-carriers or micro-carriers. In some embodiments, the pharmaceutical composition of the invention is an injectable composition e.g. a composition for injection e.g. for intramuscular injection or intravenous injection or infusion.

Typical the pharmaceutical composition may be in the form of a liquid composition ready to be injected, in the form of a concentrated liquid composition to be diluted before injection, or in the form of a powder e.g. a freeze-dried powder which is to be dissolved or suspended in an appropriate vehicle just before being administered to the subject.

The prodrug of the invention may be formulated into liposomal composition, lipid complex composition, e.g. by using excipients such as phospholipids, cholesterol and the like lipid complex or colloidal dispersion, e.g. by using surfactants and/or lipid such as those present in Abelcet® or Ambisome® formulation.

The invention also relates to a pharmaceutical kit comprising a prodrug of the invention or a pharmaceutical composition of the invention in combination with means for administration to a subject such as reconstitution buffer and/or means for injection, e.g. needle(s) and syringe(s). The kit may also include instructions for practicing the therapeutic method of the invention. Further aspects and advantages of the present invention are disclosed in the following experimental section, which should be regarded as illustrative and not limiting the scope of the present application.

### EXAMPLES

All reagents, including enzyme samples, were purchased from various commercial suppliers (Sigma Aldrich®, Fluka®, Alfa Aesar®, Acros® or TCI Chemical®) and stored according to the detailed specifications. The following solvents and reagents were freshly distilled under argon just before their use: DCM, MeCN and Et₃N over anhydrous calcium hydride; MeOH over sodium and THF over sodium and benzophenone. DCM was also sometimes purified by a Solvent Purification System (SPS). DMF was purchased anhydrous from Sigma Aldrich®. If necessary, solvents for work-up and purification were previously distilled on a Buchi R-220-SE rotavapor to remove the stabilizers.

### Example 1: Synthesis of a Prodrug of the Invention

The AmB prodrug of FIG. 1A was prepared according to the synthesis process described in FIG. 2. After optimization of reaction conditions, the AmB prodrug was achieved in a 6-step sequence with an overall yield of 58% (FIG. 2). The synthesis protocols are described here below.

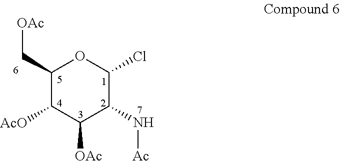 [SMILES: CC(=O)N[C@@H]1[C@@H](OC(C)=O)[C@H](OC(C)=O)[C@@H](COC(C)=O)O[C@@H]1Cl]

In a sealed tube, commercially available N-Acetyl-D-glucosamine (3.000 g, 13.50 mmol, 1.0 eq) was dissolved in freshly prepared solution of acetyl chloride saturated in HCl_{g} gas (15 mL, 210.2 mmol, 9.3 eq) and the solution was cooled down to 0° C. The reaction mixture was warmed up and stirred at room temperature for 7 d. After completion, the reaction mixture was dissolved with DCM (20 mL) and cooled down to 0° C. The organic layer was washed carefully with a saturated aqueous solution of NaHCO₃ (3×30 mL) and brine (30 mL). The organic layer was separated, dried over Na₂SO₄, filtrated and evaporated under reduced pressure. The crude product was purified by flash column chromatography on silica gel (gradient elution 100:0 to 0:100 DCM/EtOAc) to give the compound 6 (3.305 g, 67%) as an air-sensitive white solid.

¹H NMR (300.13 MHz, CDCl₃, 298.15 K): δ_{H} 6.19 (d, J₁₋₂=3.6 Hz, 1H, H¹), 5.79 (d, J₇₋₂=8.7 Hz, 1H, H⁷), 5.30 (m, 1H, H³), 5.22 (t, J_{4-3, 4-5}=9.6 Hz, 1H, H⁴), 4.57-4.50 (m, 1H, H²), 4.32-4.24 (m, 2H, H⁵, H^{6a}), 4.14 (m, 1H, H^{6b}), 2.11 (s, 3H, H^{Acetyl}), 2.06 (s, 3H, H^{Acetyl}), 2.05 (s, 3H, H^{Acetyl}), 1.99 (s, 3H, H^{Acetyl}) ppm.

¹³C NMR (75.48 MHz, CDCl₃, 298.15 K): δ_{C} 171.5 (s, C^{Acetyl}), 170.6 (s, C^{Acetyl}), 170.2 (s, C^{Acetyl}), 169.2 (s, C^{Acetyl}), 93.6 (s, C¹), 70.9 (s, C⁵), 70.1 (s, C³), 66.9 (s, C⁴), 61.1 (s, C⁶), 53.5 (s, C²), 23.1 (s, C^{Acetyl}), 21.5 (s, C^{Acetyl}), 20.7 (s, C^{Acetyl}), 20.6 (s, C^{Acetyl}) ppm.

| | |
|---|---|
| White solid | [α]_{D}²⁰: +120.4 (c 1.00, CHCl₃) |
| C₁₄H₂₀ClNO₈ | HRMS (ESI⁺): m/z calculated for |
| MW: 365.76 g · mol⁻¹ | C₁₄H₂₁ClNO₈ [M + H]⁺ 366.0956, found |
| Rr: 0.63 (EtOAc) | 366.0950 |
| mp: 122° C. | FT-IR (ATR): 1739, 1659, 1541, 1348, |
| Yield: 67% | 1207, 1033, 860, 729, 593 cm⁻¹ |

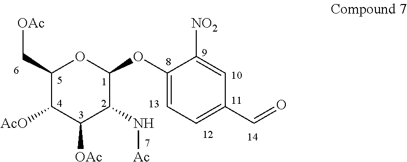 [SMILES: CC(=O)N[C@H]1[C@H](Oc2ccc(C=O)cc2[N+](=O)[O-])O[C@H](COC(C)=O)[C@@H](OC(C)=O)[C@@H]1OC(C)=O]

Commercially available compound 4-hydroxy-3-nitrobenzaldehyde (1.372 g, 8.21 mmol, 1.5 eq) was dissolved in freshly distilled MeCN (40 mL). At room temperature, activated molecular sieve 4 Å (1.000 g) and Ag₂O (2.535 g, 10.94 mmol, 2.0 eq) were added. The reaction mixture was stirred at room temperature for 15 min under positive argon atmosphere. Compound 6 (2.000 g, 5.47 mmol, 1.0 eq) was added. The reaction mixture was stirred protected from light at room temperature for 18 h under positive argon atmosphere and monitored by TLC (EtOAc, revealed with UV_{254 nm}/cerium molybdate). After completion, the reaction mixture was filtrated over a pad of celite, the residue washed with DCM and the organic layer was evaporated under reduced pressure. The crude product was purified by flash column chromatography on silica gel (gradient elution 100:0 to 0:100 DCM/EtOAc) to give the compound 7 (3.720 g, quant.) as a white solid.

¹H NMR (300.13 MHz, CDCl₃, 298.15 K): δ_{H} 9.97 (s, 1H, H¹⁴), 8.29 (s, 1H, H¹⁰), 8.05 (dd, J₁₂₋₁₀=2.0 Hz, J₁₂₋₁₃=8.2 Hz, 1H, H¹²), 7.49 (d, J₁₃₋₁₂=8.2 Hz, 1H, H¹³), 5.94 (d, J₇₋₂=6.5 Hz, 1H, H⁷), 5.81 (d, J₁₋₂=7.5 Hz, 1H, H¹), 5.70 (t, J_{3-2, 3-4}=9.6 Hz, 1H, H³), 5.13 (t, J_{4-3, 4-5}=9.2 Hz, 1H, H⁴), 4.34-3.17 (m, 2H, H⁵, H^{6a}), 4.00 (m, 1H, H^{6b}), 3.82 (m, 1H, H²), 2.10 (s, 3H, H^{Acetyl}), 2.07 (s, 3H, H^{Acetyl}), 2.06 (s, 3H, H^{Acetyl}), 1.97 (s, 3H, H^{Acetyl}) ppm.

¹³C NMR (75.48 MHz, CDCl₃, 298.15 K): δ_{C} 188.7 (s, C¹⁴), 171.3 (s, C^{Acetyl}), 170.6 (s, C^{Acetyl}), 170.4 (s, C^{Acetyl}), 169.6 (s, C^{Acetyl}), 153.8 (s, C⁸), 141.4 (s, C⁹), 134.4 (s, C¹²), 131.5 (s, C¹¹), 126.8 (s, C¹⁰), 119.5 (s, C¹³), 98.6 (s, C¹), 72.6 (s, C⁵), 70.7 (s, C³), 68.5 (s, C⁴), 62.0 (s, C⁶), 55.7 (s, C²), 23.4 (s, C^{Acetyl}), 20.8 (overlap, C^{Acetyl}, C^{Acetyl}, C^{Acetyl}) ppm.

| | |
|---|---|
| White solid | [α]_{D}²⁰: +0.50 (c 1.00, CHCl₃) |
| C₂₁H₂₄N₂O₁₂ | HRMS (ESI⁺): m/z calculated for |
| MW: 496.43 g · mol⁻¹ | C₂₁H₂₅N₂O₁₂ [M + H]⁺ 497.1390, found |
| Rf: 0.45 (EtOAc) | 497.1407 |
| mp: 165° C. | FT-IR (ATR): 1740, 1217, 1031 cm⁻¹ |
| Yield: quant. | |

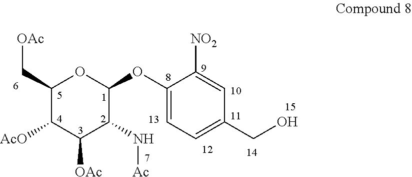 [SMILES: CC(=O)N[C@H]1[C@H](Oc2ccc(CO)cc2[N+](=O)[O-])O[C@H](COC(C)=O)[C@@H](OC(C)=O)[C@@H]1OC(C)=O]

Compound 7 (4.719 g, 9.51 mmol, 1.0 eq) was dissolved in a mixture of CHCl₃ (74 mL), i-PrOH (21 mL) and silica gel (7.608 g) and the solution was cooled down to 0° C. NaBH₄ (1.079 g, 28.53 mmol, 3.0 eq) was added and the reaction mixture was stirred at 0° C. for 15 min under positive argon atmosphere. The reaction mixture was warmed up, stirred at room temperature for 10 h under positive argon atmosphere and monitored by TLC (EtOAc, revealed with UV_{254 nm}/cerium molybdate). After completion, the reaction mixture was cooled down to 0° C. The organic layer was washed carefully with a solution of HCl at 1.0 M (15 mL) and brine (30 mL). The organic layer was separated, dried over Na₂SO₄, filtrated and evaporated under reduced pressure to give the compound 8 (5.055 g, quant.) as a white solid.

¹H NMR (300.13 MHz, CDCl₃, 298.15 K): δ_{H} 147.78 (d, J₁₀₋₁₂=2.1 Hz, 1H, H¹⁰), 7.46 (dd, J₁₂₋₁₀=2.1 Hz, J₁₂₋₁₃=8.6 Hz, 1H, H¹²), 7.35 (d, J₁₃₋₁₂=8.5 Hz, 1H, H¹³), 5.85 (d, J₇₋₂=8.2 Hz, 1H, H⁷), 5.55 (dd, J_{3-2, 3-4}=9.2, 10.4 Hz, 1H, H³), 5.45 (d, J₁₋₂=8.2 Hz, 1H, H¹), 5.12 (t, J_{4-3, 4-5}=9.5 Hz, 1H, H⁴), 4.71 (s, 2H, H¹⁴), 4.27 (dd, J₆ₐ₋₅=5.2 Hz, J_{6a-6b}=12.3 Hz, 1H, H^{6a}), 4.20 (dd, J_{6b-5}=2.7 Hz, J_{6b-6a}=12.2 Hz, 1H, H^{6b}), 3.93 (dt, J_{2-1, 2-3, 2-7}=8.1, 10.4 Hz, 1H, H²), 3.86 (m, 1H, H⁵), 2.67 (s, 1H, H¹⁵), 2.09 (s, 3H, H^{Acetyl}), 2.06 (s, 3H, H^{Acetyl}), 2.04 (s, 3H, H^{Acetyl}), 1.98 (s, 3H, H^{Acetyl}) ppm.

¹³C NMR (75.48 MHz, CDCl₃, 298.15 K): δ_{C} 171.3 (s, C^{Acetyl}), 170.7 (s, C^{Acetyl}), 170.6 (s, C^{Acetyl}), 169.6 (s, C^{Acetyl}), 148.5 (s, C⁸), 141.8 (s, C⁹), 137.6 (s, C¹¹), 132.0 (s, C¹²), 123.0 (s, C¹⁰), 121.5 (s, C¹³), 100.0 (s, C¹), 72.4 (s, C⁵), 71.5 (s, C³), 68.8 (s, C⁴), 63.6 (s, C¹⁴), 62.1 (s, C⁶), 55.4 (s, C²), 23.4 (s, C^{Acetyl}), 20.9 (s, C^{Acetyl}), 20.8 (s, C^{Acetyl}), 20.8 (s, C^{Acetyl}) ppm.

| | |
|---|---|
| White solid | [α]_{D}²⁰: +0.52 (c 1.00, CHCl₃) |
| C₂₁H₂₆N₂O₁₂ | HRMS (ESI⁺): m/z calculated for |
| MW: 498.44 g · mol⁻¹ | C₂₁H₂₆N₂O₁₂Na [M + Na]⁺ 521.1383, found |
| Rf: 0.28 (EtOAc) | 521.1389 |
| mp: 186° C. | FT-IR (ATR): 1745, 1661, 1533, 1364, |
| Yield: quant. | 1032, 751 cm⁻¹ |

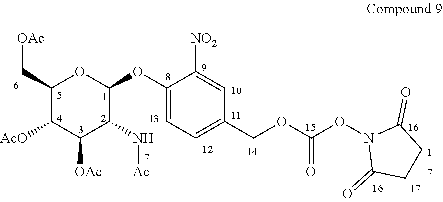 [SMILES: CC(=O)N[C@H]1[C@H](Oc2ccc(COC(=O)ON3C(=O)CCC3=O)cc2[N+](=O)[O-])O[C@H](COC(C)=O)[C@@H](OC(C)=O)[C@@H]1OC(C)=O]

Compound 8 (1.317 g, 2.64 mmol, 1.0 eq) was dissolved in freshly distilled MeCN (20 mL). Et₃N (440 μL, 3.17 mmol, 1.2 eq) and commercially available N,N′-disuccinimidyl carbonate (743 mg, 2.90 mmol, 1.1 eq) were added. The reaction mixture was stirred at room temperature for 24 h under positive argon atmosphere and monitored by TLC (EtOAc, revealed with UV_{254 nm}/cerium molybdate). After completion, the organic layer was evaporated under reduced pressure to give the crude product 9 as a highly air-sensitive yellow solid which was immediately used in the subsequent step due to its instability. To perform further characterization, once, a batch of crude product was purified by flash column chromatography on silica gel (gradient elution 100:0 to 0:100 DCM/EtOAc) to give the pure compound 9 as a highly air-sensitive white solid.

¹H NMR (300.13 MHz, CDCl₃, 298.15 K): δ_{H} 7.82 (s, 1H, H¹⁰), 7.57 (d, J₁₂₋₁₃=8.5 Hz, 1H, H¹²), 7.40 (d, J₁₃₋₁₂=8.4 Hz, 1H, H¹³), 6.58 (d, J₇₋₂=8.2 Hz, 1H, H⁷), 5.56 (d, J₁₋₂=9.0 Hz, 1H, H¹), 5.49 (d, J=10.1 Hz, 1H, H³), 5.28 (s, 2H, H¹⁴), 5.10 (t, J_{4-3, 4-5}=9.5 Hz, 1H, H⁴), 4.32-4.15 (m, 2H, H^{6a}, H^{6b}), 4.04-3.92 (m, 2H, H², H⁵), 2.84 (s, 4H, H¹⁷), 2.08 (s, 3H, H^{Acetyl}), 2.03 (s, 6H, H^{Acetyl}), 1.95 (s, 1H, H^{Acetyl}) ppm.

¹³C NMR (75.48 MHz, CDCl₃, 298.15 K): δ_{C} 172.7 (s, C^{Acetyl}), 170.9 (s, C^{Acetyl}), 169.7 (s, C^{Acetyl}) 168.9 (s, C¹⁶), 151.5 (s, C¹⁵), 150.1 (s, C⁸), 141.0 (s, C⁹), 134.2 (s, C¹²), 129.1 (s, C¹¹), 125.6 (s, C¹⁰), 120.3 (s, C¹³), 99.2 (s, C¹), 72.2 (s, C⁵), 71.3 (s, C³), 70.8 (s, C¹⁴), 68.6 (s, C⁴), 62.0 (s, C⁶), 55.0 (s, C²), 25.5 (s, C¹⁷), 23.0 (s, C^{Acetyl}), 20.9 (s, C^{Acetyl}), 20.8 (s, C^{Acetyl}) ppm.

| | |
|---|---|
| White solid | HRMS (ESI⁺): m/z calculated for |
| C₂₆H₂₉N₃O₁₆ | C₂₆H₂₉N₃O₁₆Na [M + Na] ⁺ 662.1446, found |
| MW: 639.52 g · mol⁻¹ | 662.1445 |
| Rr: 0.42 (EtOAc) | FT-IR (ATR): 1706, 1534, 1369, 1034, 648 |
| | cm⁻¹ |

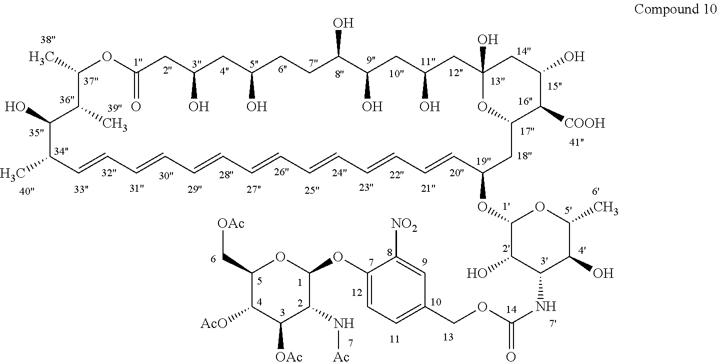 [SMILES: CC(=O)N[C@H]1[C@H](Oc2ccc(COC(=O)N[C@@H]3[C@H](O)[C@H](O[C@H]4/C=C/C=C/C=C/C=C/C=C/C=C/C=C/[C@H](C)[C@@H](O)[C@@H](C)[C@H](C)OC(=O)C[C@H](O)C[C@H](O)CC[C@@H](O)[C@H](O)C[C@H](O)C[C@]5(O)C[C@H](O)[C@@H](C(=O)O)[C@H](C4)O5)O[C@H](C)[C@H]3O)cc2[N+](=O)[O-])O[C@H](COC(C)=O)[C@@H](OC(C)=O)[C@@H]1OC(C)=O]

Crude compound 9 (1.040 g, 1.25 mmol, 5.0 eq) was dissolved in anhydrous DMF (5 mL) and the solution was stirred at room temperature for 15 min under positive argon atmosphere. Commercially available compound amphotericin B (231 mg, 0.25 mmol, 1.0 eq) and Et₃N (77 μL, 0.55 mmol, 2.2 eq) were added. The reaction mixture was stirred protected from light at room temperature for 23 h and monitored by inverse phase TLC (15:85 H₂O/organic mixture composed of 43:20 MeOH/MeCN, revealed with UV_{254 nm}/cerium molybdate). After completion, the reaction mixture was co-evaporated with toluene. The crude product was dissolved in toluene under ultrasound and placed at −18° C. The precipitate was filtrated, washed with DCM and dried under reduced pressure to give the compound 10 (312 mg, 86%) as a brown solid.

¹H NMR (300.13 MHz, 2:1 DMSO-d₆/MeOD, 298.15 K): δ_{H} 7.81 (d, J=1.7 Hz, 1H, H⁹), 7.67-7.58 (m, 1H, H¹¹), 7.41 (d, J=8.7 Hz, 1H, H¹²), 7.23-7.05 (m, 2H, H⁷, H^{T}), 6.51-5.83 (m, 12H, H^{21″} to H^{32″}), 5.47-5.32 (m, 2H, H¹, H^{33″}), 5.28-5.13 (m, 2H, H^{37″}, H⁴), 5.02 (s, 2H, H¹³), 4.95 (t J=9.5 Hz, 1H, H⁵), 4.45-4.35 (m, 2H, H^{1′}, H³), 4.35-3.92 (m, 9H, H², H^{6a}, H^{6b}, H^{5′}, H^{3″}, H^{15″}, H^{17″}, H^{19″}), 3.65-3.51 (m, 2H, H^{2′}, H^{5″}), 3.51-3.36 (m, 2H, H^{3′}, H^{4′}), 3.26-3.12 (m, 2H, H^{8″}, H^{9″}), 3.12-2.99 (m, 1H, H^{35″}), 2.36-2.24 (m, 1H, H^{34″}), 2.24-2.06 (m, 2H, H^{2″}), 2.00 (overlap, 4H, H^{Acetyl}, H^{16″}), 1.96 (s, 3H, H^{Acetyl}), 1.91 (s, 3H, H^{Acetyl}), 1.78 (s, 3H, H^{Acetyl}), 1.75-1.18 (m, 15H, H^{4″}, H^{6″}, H^{7″}, H^{10″}, H^{12″}, H^{14″}, H^{18″}, H^{36″}), 1.16 (d, J=5.5 Hz, 3H, H^{6′}), 1.10 (d, J=6.3 Hz, 3H, H^{38″}), 1.02 (d, J=6.2 Hz, 3H, H^{40″}), 0.90 (d, J=7.1 Hz, 3H, H^{39″}) ppm.

¹³C NMR (75.48 MHz, 2:1 DMSO-d₆/MeOD, 298.15 K): 6c 171.4 (s, C^{1″}), 170.8 (s, C^{Acetyl}), 170.7 (s, C^{Acetyl}), 170.4 (s, C^{Acetyl}), 169.9 (s, C^{Acetyl}), 156.5 (s, C¹⁴), 148.9 (s, C⁷), 141.2 (s, C⁸), 124.2 (s, C⁹), 137.2 (s, C^{33″}), 137.1 (s, C^{ethylenic}, 134.4 (s, C^{ethylenic}, 134.3 (s, C^{ethylenic}, 133.9 (s, C^{ethylenic}, 133.8 (s, C^{ethylenic}, 133.6 (s, C¹¹), 133.2 (m, C¹⁰, C^{ethylenic}), 133.0 (s, C^{ethylenic}), 132.9 (s, C^{ethylenic}), 132.7 (s, C^{ethylenic}), 132.6 (s, C^{ethylenic}), 132.0 (m, C^{ethylenic}, C^{ethylenic}), 129.6 (s, C^{ethylenic}, 118.3 (s, C¹²), 99.5 (s, C¹), 97.9 (s, C^{11″}), 97.5 (s, C^{1′}), 78.0 (s, C^{35″}), 75.7 (s, C³), 74.9 (s, C^{4′}), 74.3 (s, C^{19″}), 72.7 (s, C⁴), 72.1 (s, C^{5′}), 70.6 (overlap, C^{5″}, C^{8″}, C^{9″}, C^{11″}), 69.9 (s, C^{2′}), 69.7 (s, C^{37″}), 67.4 (s, C^{3″}), 66.1 (overlap, C^{15″}, C^{17″}), 64.4 (s, C¹³), 62.1 (s, C⁶), 57.5 (overlap, C^{3′}, C^{16″}), 53.7 (s, C²), 46.8 (s, C^{14″}), 44.7 (overlap, C⁴″, C^{10″}, C^{12″}), 43.2 (s, C^{34″}), 42.4 (s, C^{2″}), 36.1 (s, C^{18″}), 35.6 (overlap, C⁶″, C⁷″, C^{18″}), 22.6 (s, C^{Acetyl}), 20.6 (s, C^{Acetyl}), 20.4 (s, C^{Acetyl}), 20.4 (s, C^{Acetyl}), 18.8 (s, C^{10″}), 18.3 (s, C^{6′}), 17.1 (s, C^{38″}), 12.3 (s, C^{39″}) ppm.

In this ¹³C NMR Assignment, Some Atoms Don't have Attribution. Carbon Signal Corresponding to C^{36″} is Overlapped by DMSO-d₆ Signal

| | |
|---|---|
| Brown solid | [α]_{D}²⁰: +1.49 (c 1.00, DMSO) |
| C₆₉H₉₇N₃O₃₀ | HRMS (ESI⁺): m/z calculated for |
| MW: 1448.53 g · mol⁻¹ | C₆₉H₉₇N₃O₃₀Na [M + Na] ⁺ |
| R_{f}: 0.41 (inverse phase 15:85 | 1470.6055, found 1470.6052 |
| H₂O/organic mixture composed | FT-IR (ATR): 1722, 1231, 1044 cm⁻¹ |
| 43:20 MeOH/MeCN) | |
| mp: 155° C. | |
| Yield : 86% | |

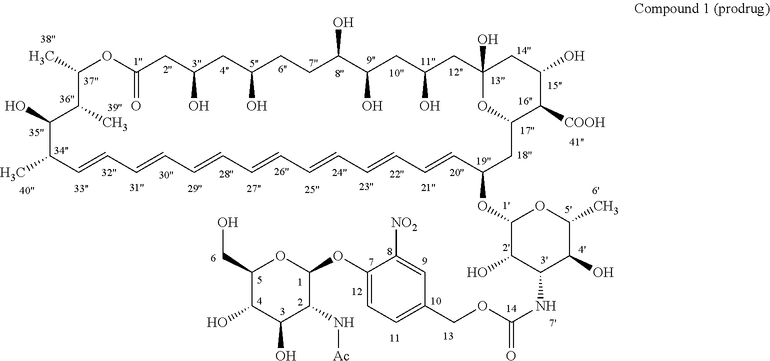 [SMILES: CC(=O)N[C@H]1[C@H](Oc2ccc(COC(=O)N[C@@H]3[C@H](O)[C@H](O[C@H]4/C=C/C=C/C=C/C=C/C=C/C=C/C=C/[C@H](C)[C@@H](O)[C@@H](C)[C@H](C)OC(=O)C[C@H](O)C[C@H](O)CC[C@@H](O)[C@H](O)C[C@H](O)C[C@]5(O)C[C@H](O)[C@@H](C(=O)O)[C@H](C4)O5)O[C@H](C)[C@H]3O)cc2[N+](=O)[O-])O[C@H](CO)[C@@H](O)[C@@H]1O]

| | |
|---|---|
| Brown solid | HRMS (ESI⁻): m/z calculated for |
| C₆₃H₉₁N₃O₂₇ | C₆₃H₉₀N₃O₂₇ [M − H]⁻ 1320.5762, |
| MW = 1322.42 g · mol⁻¹ | found 1320.5820 |
| R_{f} = 0.37 (inverse phase 15:85 | FT-IR (ATR): 3250, 1559, 1401, |
| H₂O/organic mixture composed | 1010 cm⁻¹ |
| 43:20 MeOH/MeCN) | t_{1/2 aq, pH 7.4, 37° C.} > 24 h |
| mp = 164° C. (decomposition) | |
| Yield: quant. | |

Compound 10 (259 mg, 0.18 mmol, 1.0 eq) was dissolved in a mixture of freshly distilled MeOH (1.8 mL) and THF (720 μL) and the solution was stirred at room temperature for 15 min under positive argon atmosphere. K₂CO₃ (124 mg, 0.90 mmol, 5.0 eq) was added. The reaction mixture was stirred protected from light at room temperature for 22 h and monitored by inverse phase TLC (15:85 H₂O/organic mixture composed of 43:20 MeOH/MeCN, revealed with UV_{254 nm}/cerium molybdate). After completion, sulfonic acidic resin was added and the reaction mixture was stirred at room temperature for 15 min. The reaction mixture was filtrated, the resin was thoroughly washed with MeOH and the organic layer was evaporated under reduced pressure to give the compound 11 (298 mg, quant.) as an orange solid. If needed, further purification was performed on LC preparative

¹H NMR (300.13 MHz, 2:1 DMSO-d₆/CD₃OD, 298.15 K): δ_{H} 7.82 (br, 1H), 7.76 (d, J=8.9 Hz, 1H), 7.62 (d, J=8.9 Hz, 1H), 7.43 (d, J=8.9 Hz, 1H), 7.29-7.10 (m, 1H), 7.88 (d, J=8.9 Hz, 1H), 6.53-6.20 (m, 10H), 6.20-6.01 (m, 4H), 5.99-5.84 (m, 2H), 5.49-5.39 (m, 1H), 5.38-5.31 (m, 1H), 5.25-5.14 (m, 2H), 5.12-5.07 (m, 1H), 5.04-4.99 (s, 1H), 4.83-4.73 (m, 3H), 4.68-4.61 (m, 2H), 4.48-4.36 (m, 3H), 4.27-4.17 (m, 2H), 4.11-3.99 (m, 2H), 2.75-2.70 (m, 1H), 2.32-2.24 (m, 2H), 2.19-2.14 (m, 1H), 2.08 (s, 1H), 1.78 (s, 3H, H^{Acetyl}), 1.75-1.18 (m, 15H), 1.15 (d, J=5.5 Hz, 3H), 1.11 (d, J=6.2 Hz, 3H), 1.03 (d, J=5.8 Hz, 3H), 0.91 (d, J=6.9 Hz, 3H) ppm.

### Example 2: Evaluation of the AmB Prodrug

Material and Methods

### Enzymatic Release and Aqueous Stability

In vitro enzymatic hydrolysis was carried out with commercial β-N-acetylhexosaminidase from Canavalia ensiformis E.C. 3.2.1.52 (22.8 units·mg⁻¹ protein, suspension in 2.5 M ammonium sulfate, pH 7.0). Using a VWR® Cooling Thermal Shake Touch and monitoring by analytical LC. Prodrug 1 was incubated with the enzyme according to the table below:

TABLE 1
conditions of the enzymatic assay

| Com- | Concen- | | | | |
|---|---|---|---|---|---|
| pound | tration | Enzyme | Medium | Co-solvent | Agitation |
| 1 | 150 μM | 0.07 U | 37° C. | 3% DMSO | 850 rpm |
| | | | pH 7.4 | | |
| | | | 0.1 M PBS | | |
| | | | (1 mL) | | |

Antifungal and Antiparasitic Activities (In Vitro)

Half maximal inhibitory concentration (IC50) required to inhibit 50% of the in vitro cell growth or viability were determined with broth microdilution method according to the European committee on antimicrobial susceptibility testing recommendations (protocols E.DEF 7.3.1 for Candida spp.) and according to a certified internal procedure to the laboratory IICiMed (for Leishmania spp.) (Le Pape, et al. Acta Parasitologica 2002, 47, 79-81).

The different strains, isolates and cell lines used are listed below:
Candida albicans clinical strain (IICiMed number CAAL93)
Candida albicans clinical strain (IICiMed number CAAL121)
Candida albicans reference strain SC5314 (IICiMed number CAAL146)
Leishmania major reference isolate MHOM/IL/81/BNI (IICiMed number LEMA1)
A549 cell line reference ATCC® CCL-185 (carcinoma epithelial cells from lung)
HeLa cell line reference

Growth or cell viability was performed on flat-bottomed microplate, firstly appreciated by visual reading and always confirmed using a Bio-Rad iMark microplate absorbance reader to measure the absorbance of the plate at 595 nm or using a resazurin salt cell viability assay with a Packard fluorocount microplate reader BF10000 with halogen light source to measure the fluorescence at 590 nm after excitation at 530 nm. Results represent the mean (±the standard error of the mean SEM if available) calculated from at least two independent experiments performed each in triplicate on one or several strains. The values are expressed with the corresponding pathogens and are represented in μM.

### Results

Enzymatic Release Assessment

The release of AmB from prodrug 1 has been confirmed by incubating the latter with the enzyme of interest. Real-time monitoring thus made it possible to visualize the release kinetics in the presence of the enzyme of interest as well as the good aqueous stability in the absence of this enzyme. The kinetic of release is shown in FIG. 3.

In Vitro Cell Assessment

The prodrug was tested on different cell types, blastospore or filamented yeasts to determine an antifungal activity, Leishmania promastigotes and intracellular amastigotes to determine an antiprotozoal activity and finally on human cells to detect cytotoxicity. The results are shown in the below table 2:

TABLE 2
Antifungal and Antiparasitic activity versus human cells cytotoxicity
(*morphology changes were observed at 10 μM)

| | |
|---|---|
| Amphotericin B | Compound 1 |

| | | | | |
|---|---|---|---|---|
| Candida albicans | 0.36 ± 0.04 | μM | 0.43 ± 0.02 | μM |
| (blastospores) | | | | |
| Candida albicans | 0.31 ± 0.01 | μM | 0.42 ± 0.01 | μM |
| (filamented yeasts) | | | | |
| Leishmania major | 0.31 ± 0.02 | μM | 0.27 ± 0.06 | μM |
| (promastigote) | | | | |
| Leishmania major | <0.10 | μM | <0.10 | μM |
| (amastigote) | | | | |
| Cryptococcus | 0.03 | μM | 0.25 | μM |
| neoformans | | | | |
| Cryptococcus gattii | 0.04 | μM | 0.60 | μM |
| HeLa cells | 23.29 ± 0.11 | μM* | >100 | μM |

| | | | |
|---|---|---|---|
| A549 | (not tested) | >100 | μM |
| hPBMC | (not tested) | >50 | μM |

Compound 1 showed the same level of activity as amphotericin B against C. albicans or L. major. Compound 1 was also showed to be active on Cryptococcus neoformans and Cryptococcus gattii. Of note, Compound 1 was less toxic against Hela cells. Furthermore, compound 1 showed no toxicity (CI₅₀>50 μM) against a pneumocyte line A549 and human PBMC.

### Example 3: Assessment of the Antifungal Activity of AmB Prodrug In Vivo

### Material and Methods

Mice were immunosuppressed by subcutaneous injection of 30 mg/kg prednisolone one day before challenge. On day 0, mice were infected intravenously C. albicans blastoconidia. One hour after infection, mice were treated intraperitoneally once daily with 1 mg/kg body weight of Fungizone®, Ambisome® and AmB prodrug (Compound 1, FIG. 1A) for 3 consecutive days. The control group received sterile distillated water (vehicle). Survival was monitored for 14 days post inoculation. Differences in cohorts were analyzed by the log-rank test. On day 14, all mice were euthanized and their kidneys were excised and weighed. Tissues were homogenized and serially diluted 10- to 1000-fold in sterile saline, then plated onto Sabouraud dextrose agar and incubated for 48 h to determine the number of CFUs. Tissue fungal burden was expressed as average log CFUs/gram of tissue. Differences in mean CFUs in kidneys were compared with the vehicle control using a one-way ANOVA with a post-hoc Tukey test. A P value of <0.05 was considered statistically significant.

Results

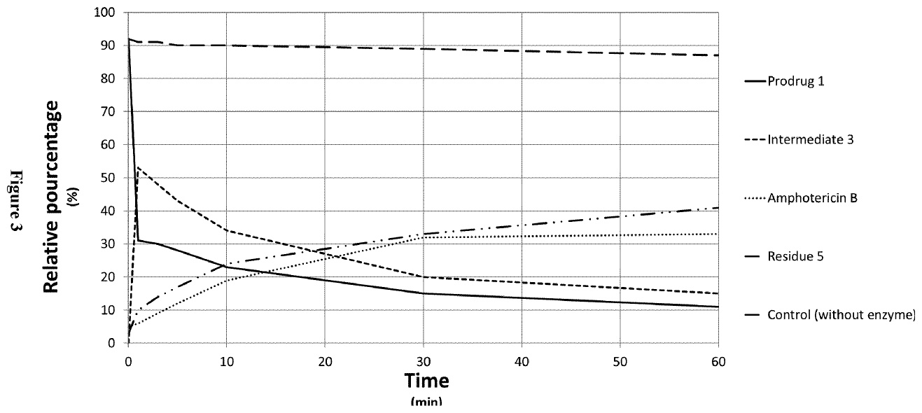

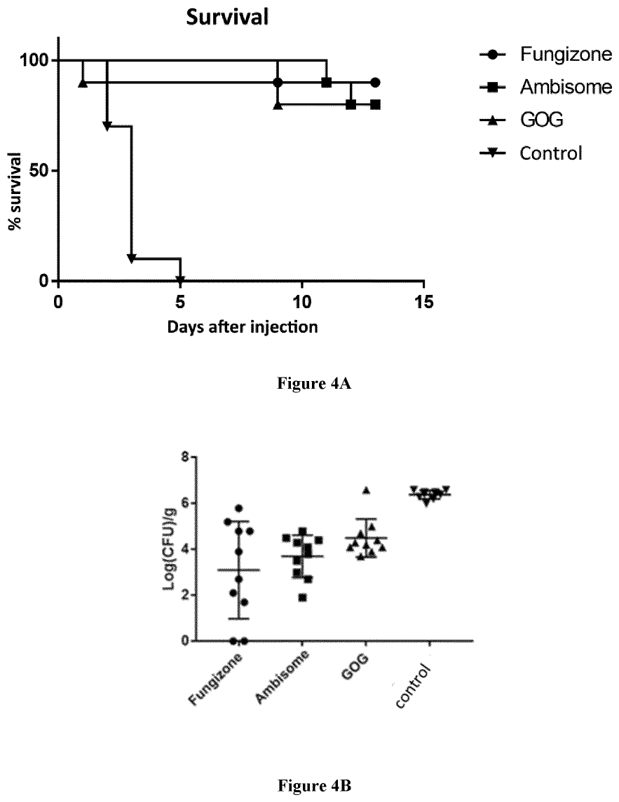

FIG. 1 showed that mice treated with vehicle died before day 5. All treatments (Fungizone® (Amphotericin B), Ambisome® (AmB in liposomal composition) and AmB prodrug of the invention (called GOG in FIGS. 4A and 4B) statically improved survival (p>0.001). No statistical difference was observed between treatments used. Regarding the fungal burden of the kidney, mice treated with the AmB prodrug showed a significantly reduction of the burden (p<0.0079) as compared to the control group administered with the placebo. No statistical difference was measured between treatments used (p>0.05). In other words, these data showed that the AmB prodrug of the invention is at least as effective as AmB drugs.

### Example 4: Assessment of the AmB Prodrug in the Galleria mellonella Model

Evaluation of Toxicity on the Galleria mellonella Model

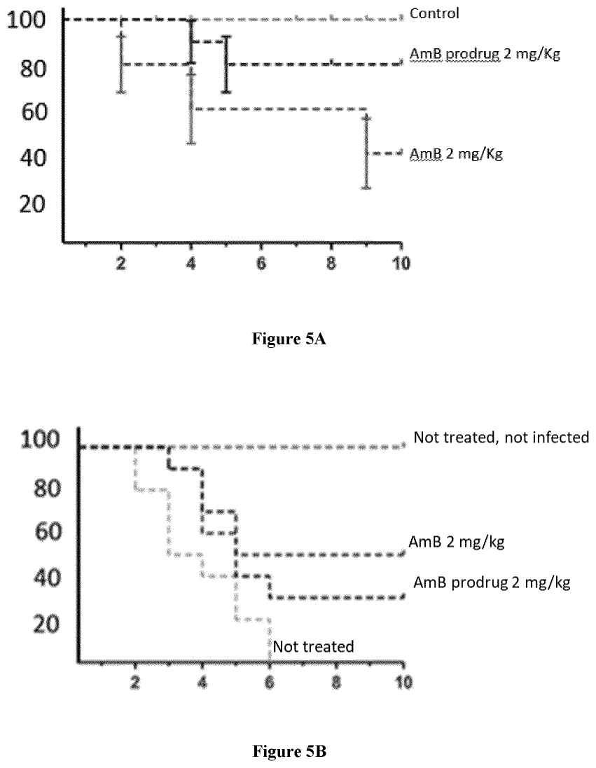

The larval model represents a rapid and practical tool for assessing the intrinsic toxicity of an active substance. See Le Pape et al, 2019, Int J Infect Dis. 2019 April; 81:85-90 for more details concerning the model. The larvae were incubated with AmB, AmB prodrug (Compound 1) and vehicle. The results concerning toxicity are shown in FIG. 5A. At the dose of 2 mg·kg-1, AmB was very toxic leading to a 40% survival of the treated group. In comparison, at an equivalent dose, its prodrug was much less toxic with a percentage survival of 80%. This statistically significant difference confirmed the in vitro results on human cells and showed the reduced toxicity of AmB in its carbamate prodrug form.

Evaluation of In Vivo Anti-Cryptococcus Activity in Galleria mellonella Model

This model also constitutes a screening model and a good alternative to the traditional studies on murine models for the evaluation of the activity of antifungal molecules. The AmB prodrug of the invention (Compound 1) was evaluated for its antifungal efficacy against Cryptococcus neoformans and Cr. gattii. FIG. 5B shows survival curves of G. mellonella infected with Cryptococcus neoformans and treated with amphotericin B or its carbamate N-acetyl-D-glucosamine prodrug. In the absence of treatment, all larvae died after 6 days and 5 days, respectively. For Cryptococcus neoformans, at a dose of 2 mg·kg-1, amphotericin B led to 50% survival and its prodrug was also effective with a percentage survival of 30% at an equivalent dose.

In the case of Cr. gattii, amphotericin B and the prodrug led to 30% and 20% survival respectively.

1-19. (canceled)

20. An antifungal prodrug of formula (A): 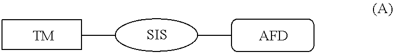
wherein:
AFD refers to an antifungal drug,
SIS refers to a self-immolative spacer which is covalently bound to AFD and to TM, and
TM refers to a trigger moiety selected from glycosyl residues and oligosaccharides, said TM stabilizes SIS and is cleavable by a pathogen hydrolytic enzyme, and
wherein when TM is cleaved by the pathogen hydrolytic enzyme, SIS undergoes a spontaneous degradation so as to release AFD.

21. The antifungal prodrug of claim 20, wherein:
TM is selected from the group consisting of hexosamines, N-acetyl hexosamines, neuraminic acid, sialic acid and oligosaccharides thereof comprising from 2 to 50 glycosyl residues; and/or
AFD is selected from the group consisting of azole antifungals, polyene antifungals, echinocandins, orotomides and enfumafungin aglycon derivatives.

22. The antifungal prodrug of claim 20, wherein TM is selected from the group consisting of glucosamine, galactosamine, mannosamine, neuraminic acid, N-acetylglucosamine, N-acetylgalactosamine, sialic acid, N-acetyl mannosamine and chitine.

23. The antifungal prodrug of claim 20, wherein TM is N-acetylglucosamine or N-acetylgalactosamine.

24. The antifungal prodrug of claim 20, wherein AFD is selected from the group consisting of amphotericin B, nystatin, natamycin, caspofungin, micafungin, anidulafungin, rezafungin, votriconazole, ketoconazole, itraconazole, fluconazole, ibrexafungerp, olorofim and derivatives thereof.

25. The antifungal prodrug of claim 24, wherein AFD is caspofungin, votriconazole or amphotericin B.

26. The antifungal prodrug of claim 20, wherein SIS is selected from self-immolative spacers which undergo spontaneous degradation involving an electronic cascade or a cyclization.

27. The antifungal prodrug of claim 20, wherein SIS comprises or consists in a moiety of formula (Ia1), (Ib1), (Ic1) or (Id1): 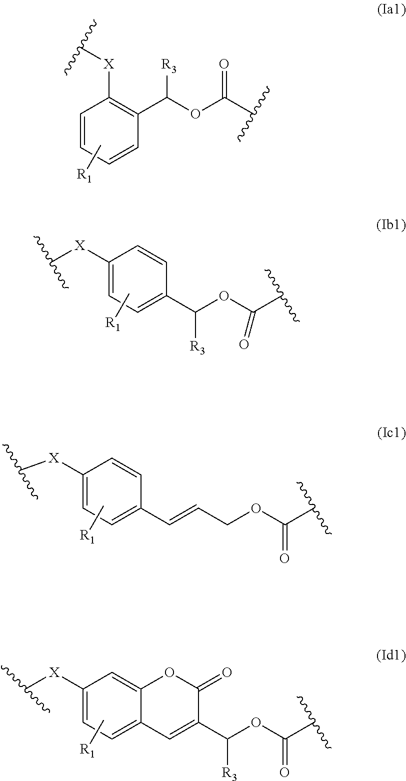
wherein:
X is O, S, —O(C═O)—NH—, O(C═O)O—, —O(P═O)O—, —O(P═S)O—, NR, with R is H or a C₁-C₃ alkyl,
R₁ is H, a halogen, —NO₂, C₁-C₃ alkyl, —CF₃, —NHR, —OR, —C(═O)OR, —SO₂R, with R is H or a C₁-C₃ alkyl, or a targeting moiety, and
R₃ is H or a targeting moiety, and
R₁ and R₃ are not a targeting moiety at the same time.

28. The antifungal prodrug of claim 27, wherein R₃ is H and R₁ is H, a halogen, —NO₂, —CF₃—OR, —C(═O)OR, —SO₂R, with R is H or a C₁-C₃ alkyl.

29. The antifungal prodrug of claim 20, which is of formula (A2): 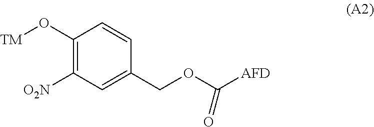
wherein:
TM is a glycosyl residue selected from the group consisting of glucosamine, galactosamine, N-acetylglucosamine, N-acetylgalactosamine, mannosamine neuraminic acid, and sialic acid, and
AFD is an antifungal drug selected from the group consisting of amphotericin B, nystatin, natamycin, caspofungin, micafungin, anidulafungin, rezafungin, votriconazole, ketoconazole, itraconazole, fluconazole and derivatives thereof.

30. The antifungal prodrug of claim 20, said prodrug being: 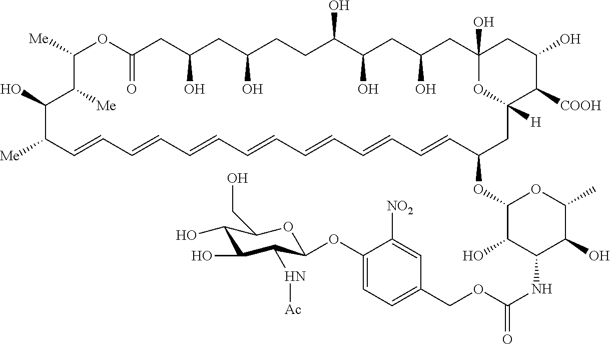
or a pharmaceutically acceptable salt thereof.

31. The antifungal prodrug of claim 20, wherein TM is cleavable by a pathogen hydrolytic enzyme which is an extracellular glycosidase (EC 3.2.1).

32. A method of treating an infectious disease comprising administering an antifungal prodrug of claim 20 to a subject in need of treatment.

33. The method of claim 32, wherein the infectious disease is caused by a pathogen belonging to Candida, Aspergillus, Cryptococcus, Mucorales, Fusarium, Scedosporium, Lomentospora, Blastomyces, Mucorales order or Leishmania, Trypanosoma, or Plasmodium species.

34. The method of claim 32, wherein the subject is immunocompromised and the infectious disease is an invasive fungal disease.

35. The method of claim 32, wherein the infectious disease is caused by a pathogen belonging to Candida, Aspergillus, Cryptococcus, Mucorales, Fusarium, Scedosporium, Lomentospora, Blastomyces, Mucorales order or Leishmania, Trypanosoma, Plasmodium species and/or the subject is immunocompromised.

36. The method of claim 32, wherein the antifungal prodrug is administered orally or intravenously.

37. The method of claim 32, wherein the antifungal prodrug is 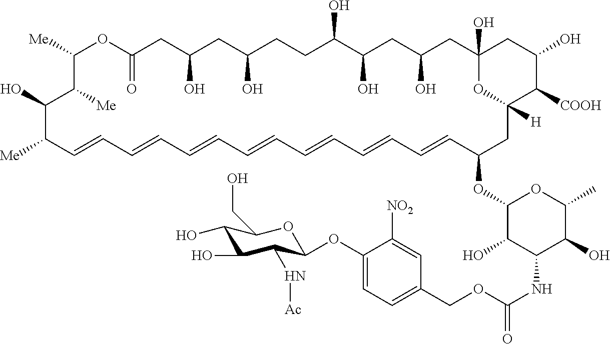
or a pharmaceutically acceptable salt thereof.

38. A pharmaceutical composition comprising the antifungal prodrug of claim 20 and a pharmaceutically acceptable excipient.
